(19) United States
(12) Patent Application Publication
(10) Pub. No.: US 2026/0103753 A1
(43) Pub. Date: Apr. 16, 2026

(54) METHODS AND COMPOSITIONS FOR NUCLEIC ACID SEQUENCING USING PREDOMINANTLY UNLABELED NUCLEOTIDES

(71) Applicant: esBiolab, LLC, San Diego, CA (US)

(72) Inventors: Wei LI, San Diego, CA (US); Liangliang QIANG, San Diego, CA (US)

(21) Appl. No.: 19/355,493

(22) Filed: Oct. 10, 2025

Related U.S. Application Data
(63) Continuation of application No. PCT/US2024/024441, filed on Apr. 12, 2024.
(60) Provisional application No. 63/459,217, filed on Apr. 13, 2023.

(51) Int. Cl. C12Q 1/6874

(52) U.S. Cl. CPC C12Q 1/6874

(57) ABSTRACT

The present disclosure relates in some aspects to methods and compositions for analyzing nucleic acid sequences, such as for determining sequences of nucleic acid molecules in a clonal cluster, including DNA sequencing methods and nucleotide compositions.

### CROSS-REFERENCE TO RELATED APPLICATIONS

This application is a continuation of International Patent Application No. PCT/US2024/024441, filed on Apr. 12, 2024, entitled “METHODS AND COMPOSITIONS FOR NUCLEIC ACID SEQUENCING USING PREDOMINANTLY UNLABELED NUCLEOTIDES,” which claims priority to U.S. Provisional Application No. 63/459,217, filed on Apr. 13, 2023, entitled “METHODS AND COMPOSITIONS FOR NUCLEIC ACID SEQUENCING USING PREDOMINANTLY UNLABELED NUCLEOTIDES,” which applications are herein incorporated by reference in their entireties for all purposes.

### FIELD

The present disclosure relates in some aspects to methods and compositions for analyzing nucleic acid sequences, such as for determining sequences of nucleic acid molecules in a clonal cluster, including DNA sequencing methods and nucleotide compositions.

### BACKGROUND

Existing methods for DNA-sequencing often use fluorescent labels to distinguish nucleotide bases. Labeled nucleotide bases, however, are inefficiently incorporated into the nucleic acid strand being synthesized, on account of the labels. Nevertheless, some existing methods dedicate a unique fluorescent label for each of the four nucleotide bases, and other existing methods use labels to denote three of the four nucleotide bases. In other words, the existing methods suffer from poor nucleotide base incorporation efficiency. Improved methods for nucleic acid sequencing are needed. Provided herein are methods, compositions, and kits that meet such and other needs.

### BRIEF SUMMARY

Disclosed herein are methods and systems that overcome nucleotide incorporation inefficiencies during nucleic acid sequencing. Existing methods for nucleic acid sequencing rely extensively on labelled nucleotides. Such nucleotides often consist of at least one fluorescent molecule conjugated to the nucleotide base, which promotes steric hindrance and inefficient reactions. Inefficient nucleotide incorporation during nucleic acid sequencing reactions can be the basis for several additional problems for nucleic acid sequencing methods. To address the issues that arise from inefficient nucleotide incorporation, in some embodiments, methods of the present disclosure employ an alternative scheme, so that unlabeled nucleotides are predominantly used or more extensively used (as compared to certain existing methods) during nucleic acid sequencing. The methods of the present disclosure address inefficient nucleotide incorporation during nucleic acid sequencing, and by extension, yield faster sequencing reaction times, fewer out-of-phase sequencing reactions, and cheaper sequencing reaction costs.

In some embodiments, provided herein is a method for determining a template sequence of a polynucleotide template at a location on a substrate, wherein multiple copies of the template sequence are provided at the location, the method comprising: a) contacting the substrate with a first pool of nucleotides comprising labeled nucleotides configured to be detected at different wavelengths; b) allowing binding and optional incorporation of the nucleotides of the first pool on a first subset of the multiple copies of the template sequence at the location, wherein the bound and optionally incorporated nucleotides are complementary to nucleotide residues at a first nucleotide position in the template sequence; and c) imaging the substrate to detect a signal (or record an absence thereof) at the location at each of the different wavelengths, wherein the signals or absences thereof are associated with a bound and optionally incorporated nucleotide of the first pool at the location. In some embodiments, the method comprises d) contacting the substrate with a second pool of nucleotides. In any of the preceding embodiments, the second pool of nucleotides can comprise nucleotides configured to be detected at a first wavelength of the different wavelengths, and optionally nucleotides configured to be detected at a second wavelength of the different wavelengths. In any of the preceding embodiments, the method can comprise e) allowing binding and optional incorporation of the nucleotides of the second pool on a second subset of the multiple copies of the template sequence at the location, wherein the first and second subsets are different subsets, and wherein the bound and optionally incorporated nucleotides are complementary to nucleotide residues at the first nucleotide position in the template sequence. In any of the preceding embodiments, the method can comprise f) imaging the substrate to detect a signal (or record an absence thereof) at the location at each of the different wavelengths, wherein the signals or absences thereof are associated with a bound and optionally incorporated nucleotide of the second pool at the location. In any of the preceding embodiments, the method can comprise generating, for the location, a signal codeword comprising signal codes corresponding to the signals or absences thereof in c) and f) directed or recorded at the location, wherein the signal codeword corresponds to the identity of the base in the bound and optionally incorporated nucleotides, thereby identifying the nucleotide residue at the first nucleotide position in the template sequence. In some embodiments, the first pool of nucleotides are used in a)-c), and the second pool of nucleotides are used in d)-f). In some embodiments, the second pool of nucleotides are used in a)-c), and the first pool of nucleotides are used in d)-f).

In any of the preceding embodiments, the polynucleotide template can be in a cluster of multiple polynucleotides immobilized at the location on the substrate. In any of the preceding embodiments, each polynucleotide in the cluster can comprise one or more copies of the template sequence. In any of the preceding embodiments, the substrate can comprise a plurality of clusters immobilized thereon. In any of the preceding embodiments, each cluster can comprise multiple polynucleotides immobilized at a spatially discrete location on the substrate. In any of the preceding embodiments, the average distance between adjacent clusters on the substrate can be between about 0.3 μm and about 10 μm. In any of the preceding embodiments, the density of clusters on the substrate can be between about 100 k/mm² and about 5000 k/mm². In any of the preceding embodiments, signals from adjacent clusters on the substrate are optically resolvable. In any of the preceding embodiments, the plurality of clusters can be part of a random array of clusters on the substrate. In any of the preceding embodiments, the plurality of clusters can be part of an ordered array of clusters on the substrate. In any of the preceding embodiments, one or more clusters of the plurality of clusters can be formed via bridge amplification. In any of the preceding embodiments, the one or more clusters each can comprise multiple molecules each comprising: i) one or more adapter sequences and/or one or more primer binding sequences and ii) the template sequence or a complement thereof.

In any of the preceding embodiments, the polynucleotide template can comprise a rolling circle amplification product immobilized on the substrate. In any of the preceding embodiments, the rolling circle amplification product can comprise multiple copies of the template sequence. In any of the preceding embodiments, the rolling circle amplification product can comprise multiple copies of a unit sequence comprising: i) one or more adapter sequences and/or one or more primer binding sequences and ii) the template sequence.

In any of the preceding embodiments, at each of the location(s), one or more polynucleotides can be 5′ immobilized on the substrate and 3′ blocked. In any of the preceding embodiments, at each of the location(s), one or more polynucleotides can be 3′ immobilized on the substrate. In any of the preceding embodiments, at each of the location(s), one or more nucleic acid concatemers can be immobilized on the substrate.

In any of the preceding embodiments, the first pool of nucleotides can comprise nucleotides of any one, two, or three of A, T/U, C, and G, or nucleotides of A, T/U, C, and G. In any of the preceding embodiments, the first pool of nucleotides can comprise i) nucleotide molecules of the same base, wherein each nucleotide molecule is labeled with or configured to be labeled with two or more different detectable labels detectable at different wavelengths; or ii) nucleotide molecules of the same base, wherein different subsets of the nucleotide molecules are each labeled with or configured to be labeled with two or more different detectable labels detectable at different wavelengths. In any of the preceding embodiments, the first pool of nucleotides can comprise labeled nucleotides configured to be detected at two different wavelengths, optionally wherein the first pool of nucleotides comprises nucleotides of one or two different bases that are not configured to be detected. In any of the preceding embodiments, the first pool of nucleotides can comprise one or more dNTP monomers and/or one or more dNTP multimers each comprising multiple dNTP molecules conjugated to a scaffold, optionally wherein the scaffold is conjugated to one or more fluorescent moieties and optionally wherein the scaffold comprises a streptavidin.

In any of the preceding embodiments, on average, no more than about 90%, about 80%, about 70%, about 60%, about 50%, about 40%, about 30%, about 20%, or about 10% of the multiple copies of the template sequence at each location on the substrate can be used as templates for the binding and optional incorporation of the nucleotides of the first pool. In any of the preceding embodiments, on average, about 50% of the multiple copies of the template sequence at each location on the substrate can be used as templates for the binding and optional incorporation of the nucleotides of the first pool.

In any of the preceding embodiments, each location on the substrate can be contacted with the first pool of nucleotides for no more than about 18, about 15, about 12, about 9, about 6, or about 3 seconds. In any of the preceding embodiments, each location on the substrate can be contacted with the first pool of nucleotides for about 2 seconds.

In any of the preceding embodiments, the first pool of nucleotides can each comprise a reversible terminator, optionally wherein the reversible terminator is a 3′-O-blocked reversible terminator or a 3′-unblocked reversible terminator. In any of the preceding embodiments, the method can comprise removing unbound or unincorporated nucleotides from the substrate prior to imaging the substrate in c). In any of the preceding embodiments, the imaging in c) can comprise imaging the substrate using two different fluorescent channels.

In any of the preceding embodiments, the second pool of nucleotides can comprise nucleotides of any one, two, or three of A, T/U, C, and G, or nucleotides of A, T/U, C, and G. In any of the preceding embodiments, the second pool of nucleotides can comprise labeled nucleotides configured to be detected at only one wavelength. In any of the preceding embodiments, the second pool of nucleotides can comprise labeled nucleotides configured to be detected at two different wavelengths. In any of the preceding embodiments, the second pool of nucleotides can comprise: i) nucleotide molecules of the same base, wherein each nucleotide molecule is labeled with or configured to be labeled with two or more different detectable labels detectable at different wavelengths; or ii) nucleotide molecules of the same base, wherein different subsets of the nucleotide molecules are each labeled with or configured to be labeled with two or more different detectable labels detectable at different wavelengths. In any of the preceding embodiments, the second pool of nucleotides can comprise nucleotides of one, two, or three different bases that are not configured to be detected. In any of the preceding embodiments, the second pool of nucleotides can comprise one or more dNTP monomers and/or one or more dNTP multimers each comprising multiple dNTP molecules conjugated to a scaffold, optionally wherein the scaffold is conjugated to one or more fluorescent moieties and optionally wherein the scaffold comprises a streptavidin.

In any of the preceding embodiments, each location on the substrate can be contacted with the second pool of nucleotides until substantially all of remaining copies of the template sequence at the location are used as templates for the binding and optional incorporation of the nucleotides of the second pool. In any of the preceding embodiments, each location on the substrate can be contacted with the second pool of nucleotides for at least about 20, about 25, or about 30 seconds or longer. In any of the preceding embodiments, each location on the substrate can be contacted with the second pool of nucleotides for about 30 seconds.

In any of the preceding embodiments, the second pool of nucleotides can each comprise a reversible terminator, optionally wherein the reversible terminator is a 3′-O-blocked reversible terminator or a 3′-unblocked reversible terminator. In any of the preceding embodiments, the method can comprise removing unbound or unincorporated nucleotides from the substrate prior to imaging the substrate in f).

In any of the preceding embodiments, the imaging in f) can comprise imaging the substrate using one or two different fluorescent channels. In any of the preceding embodiments, the method can comprise washing the substrate for one or more times between c) and d).

In any of the preceding embodiments, a different signal codeword can be generated for each of a plurality of the different locations on the substrate, each different signal codeword corresponding to one of A, T/U, C, and G. In any of the preceding embodiments, the method can comprise determining the bases at a second nucleotide position in the template sequence, wherein the second nucleotide position is 5′ to the first nucleotide position in the template sequence.

In any of the preceding embodiments, one or more nucleotides in the first pool and/or one or more nucleotides in the second pool can be each fluorescently labeled with one or more fluorophores prior to the binding and optional incorporation templated on the template sequence; or, one or more nucleotides in the first pool and/or one or more nucleotides in the second pool can be each labeled with a binding moiety prior to the binding and optional incorporation templated on the template sequence, and a fluorescently labeled binder can be attached to the binding moiety after the binding and optional incorporation and prior to the imaging. In any of the preceding embodiments, the binding moiety/fluorescently labeled binder pair can comprise biotin, DNP, DIG, or desthiobiotin. In any of the preceding embodiments, the binding moiety/fluorescently labeled binder pair can comprise functional groups capable of reacting with each other, optionally wherein the functional groups are click functional groups. In any of the preceding embodiments, the binding moieties in the nucleotides of the first and/or second pools can be non-fluorescent, and the nucleotides of the first and/or second pools can become fluorescently detectable after the fluorescently labeled binders are attached to the corresponding binding moieties. In any of the preceding embodiments, the attachment of the fluorescently labeled binder to the binding moiety can be reversible. In any of the preceding embodiments, each fluorescent label can be removable (e.g., cleavable) from the nucleotide attached thereto.

In any of the preceding embodiments, the first pool of nucleotides can comprise nucleotides of A, T/U, C, and G, and nucleotides of two of the four different bases can comprise nucleotides labeled with a label detectable at a first wavelength, and nucleotides of the remaining two of the four different bases can comprise nucleotides labeled with a label detectable at a second wavelength different from the first wavelength.

In any of the preceding embodiments, the second pool of nucleotides can comprise nucleotides of A, T/U, C, and G, and nucleotides of one of the four different bases can comprise nucleotides labeled with a label detectable at a first wavelength, and nucleotides of a different one of the four different bases can comprise nucleotides labeled with a label detectable at a second wavelength different from the first wavelength, and nucleotides of the remaining two of the four different bases are not detectably labeled.

In any of the preceding embodiments, the bound and optionally incorporated nucleotide of the first pool at the location in c) can remain detectable in f). In any of the preceding embodiments, in f), the bound and optionally incorporated nucleotide of the first pool at the location can be detected at one or more of the different wavelengths. In any of the preceding embodiments, in c), the nucleotide of the first pool can be incorporated at the location, and in f), the nucleotide of the second pool can be bound and optionally incorporated at the location.

In some embodiments, provided herein is a method for determining sequences of a plurality of polynucleotide templates having different template sequences, comprising: a) contacting a substrate having clusters of polynucleotides immobilized thereon with a first pool of nucleotides, wherein each cluster comprises: i) multiple copies of one of the different template sequences, and ii) an oligonucleotide primer annealed to a primer binding sequence for extension of the oligonucleotide primer templated on a copy of the template sequence of i), wherein the first pool of nucleotides comprises: i) at least two different bases, and ii) labeled nucleotides configured to be detected at different wavelengths; b) allowing binding and optional incorporation of the nucleotides of the first pool templated on only a subset of the multiple copies of the template sequence at each cluster, wherein the bound and optionally incorporated nucleotides are complementary to nucleotides at a first nucleotide position in the template sequences; c) imaging the substrate to detect signals or absence thereof at the clusters, wherein the signals are associated with the bound and optionally incorporated nucleotides of the first pool; d) contacting the substrate with a second pool of nucleotides comprising: i) at least two different bases, and ii) labeled nucleotides configured to be detected at one or more wavelengths; e) allowing binding and optional incorporation of the nucleotides of the second pool templated on the template sequence at each cluster, wherein the bound and optionally incorporated nucleotides are complementary to nucleotides at the first nucleotide position in the template sequences; and f) imaging the substrate to detect signals or absence thereof at the clusters, wherein the signals are associated with the bound and optionally incorporated nucleotides of the second pool, wherein for each cluster, a signal codeword comprising signal codes corresponding to the signals or absence thereof detected in c) and f) is generated, wherein different signal codewords correspond to different bases, thereby determining the bases at the first nucleotide position in the plurality of polynucleotide templates.

In some embodiments, the clusters are disposed at spatially discrete sites on the substrate. In any of the embodiments herein, the average distance between adjacent clusters on the substrate can be between about 0.3 μm and about 10 μm. In any of the embodiments herein, the density of clusters on the substrate can be between about 100 k/mm² and about 5000 k/mm².

In any of the embodiments herein, signals from adjacent clusters on the substrate can be optically resolvable. In any of the embodiments herein, the clusters can comprise a random array of clusters on the substrate. In any of the embodiments herein, the clusters can comprise an ordered array of clusters on the substrate. In any of the embodiments herein, one or more of the clusters can be formed via bridge amplification. In any of the embodiments herein, the one or more clusters can each comprise multiple molecules each comprising: i) one or more adapter sequences and/or one or more primer binding sequences and ii) the same template sequence or complement thereof. In any of the embodiments herein, one or more of the clusters can be formed via rolling circle amplification (RCA).

In any of the embodiments herein, the one or more clusters can each comprise one or more RCA products (RCPs) each comprising: i) one or more adapter sequences and/or one or more primer binding sequences and ii) the same template sequence. In any of the embodiments herein, each cluster can comprise: i) one or more polynucleotides that are 5′ immobilized on the substrate and 3′ blocked; ii) one or more polynucleotides that are 3′ immobilized on the substrate; and/or iii) one or more nucleic acid concatemers immobilized on the substrate.

In any of the embodiments herein, the first pool of nucleotides can comprise nucleotides of any two or three of A, T/U, C, and G. In any of the embodiments herein, the first pool of nucleotides comprises nucleotides of A, T/U, C, and G. In any of the embodiments herein, the first pool of nucleotides can comprise: i) nucleotide molecules of the same base each labeled with or configured to be labeled with two or more different detectable labels; or ii) nucleotide molecules of the same base, wherein different subsets of the nucleotide molecules are each labeled with or configured to be labeled with two or more different detectable labels. In any of the embodiments herein, the first pool of nucleotides can comprise labeled nucleotides configured to be detected at two different wavelengths, optionally wherein the first pool of nucleotides comprises nucleotides of one or two different bases that are not configured to be detected. In any of the embodiments herein, the first pool of nucleotides can comprise one or more dNTP monomers and/or one or more dNTP multimers each comprising multiple dNTP molecules conjugated to a scaffold. In any of the embodiments herein, the scaffold can be conjugated to one or more fluorescent moieties. In any of the embodiments herein, the scaffold can comprise a streptavidin.

In any of the embodiments herein, on average, no more than about 90%, about 80%, about 70%, about 60%, about 50%, about 40%, about 30%, about 20%, or about 10% of the multiple copies of the template sequence at each cluster can be used as templates for the binding and optional incorporation of the nucleotides of the first pool. In any of the embodiments herein, on average about 50% of the multiple copies of the template sequence at each cluster can be used as templates for the binding and optional incorporation of the nucleotides of the first pool.

In any of the embodiments herein, the clusters can be allowed to contact with the first pool of nucleotides for no more than about 18, about 15, about 12, about 9, about 6, or about 3 seconds. In any of the embodiments herein, the clusters can be allowed to contact with the first pool of nucleotides for about 2 seconds. In any of the embodiments herein, the first pool of nucleotides can each comprise a reversible terminator, optionally wherein the reversible terminator is a 3′-O-blocked reversible terminator or a 3′-unblocked reversible terminator. In any of the embodiments herein, the embodiment can comprise removing unbound or unincorporated nucleotides from the substrate prior to imaging the substrate in c). In any of the embodiments herein, the imaging in c) can comprise imaging the substrate using two different fluorescent channels.

In any of the embodiments herein, the second pool of nucleotides can comprise nucleotides of any two or three of A, T/U, C, and G. In any of the embodiments herein, the second pool of nucleotides can comprise nucleotides of A, T/U, C, and G. In any of the embodiments herein, the second pool of nucleotides can comprise labeled nucleotides configured to be detected at two different wavelengths. In any of the embodiments herein, the second pool of nucleotides can comprise: i) nucleotide molecules of the same base each labeled with or configured to be labeled with two or more different detectable labels; or ii) nucleotide molecules of the same base, wherein different subsets of the nucleotide molecules are each labeled with or configured to be labeled with two or more different detectable labels. In any of the embodiments herein, the second pool of nucleotides can comprise nucleotides of one, two, or three different bases that are not configured to be detected. In any of the embodiments herein, the second pool of nucleotides can comprise one or more dNTP monomers and/or one or more dNTP multimers each comprising multiple dNTP molecules conjugated to a scaffold. In any of the embodiments herein, the scaffold can be conjugated to one or more fluorescent moieties. In any of the embodiments herein, the scaffold can comprise a streptavidin.

In any of the embodiments herein, the clusters can be allowed to contact with the second pool of nucleotides until substantially all of remaining copies of the template sequence at each cluster are used as templates for the binding and optional incorporation of the nucleotides of the second pool. In any of the embodiments herein, the clusters can be allowed to contact with the second pool of nucleotides for at least about 20, about 25, or about 30 seconds or longer. In any of the embodiments herein, the clusters can be allowed to contact with the second pool of nucleotides for about 30 seconds. In any of the embodiments herein, the second pool of nucleotides can each comprise a reversible terminator, optionally wherein the reversible terminator is a 3′-O-blocked reversible terminator or a 3′-unblocked reversible terminator. In any of the embodiments herein, the methods can comprise removing unbound or unincorporated nucleotides from the substrate prior to imaging the substrate in f).

In any of the embodiments herein, the imaging in f) can comprise imaging the substrate using one or two different fluorescent channels. In any of the embodiments herein, in g), each different signal codeword can correspond to one of A, T/U, C, and G. In any of the embodiments herein, the methods can comprise determining the bases at a second nucleotide position in the plurality of polynucleotide templates, wherein the second nucleotide position is 5′ to the first nucleotide position in the plurality of polynucleotide templates.

In any of the embodiments herein, the labeled nucleotides in the first pool and/or the labeled nucleotides in the second pool can be fluorescently labeled with one or more fluorophores prior to the binding and optional incorporation of the labeled nucleotides templated on the template sequences. In any of the embodiments herein, the labeled nucleotides in the first pool and/or the labeled nucleotides in the second pool can each be labeled with a binding moiety prior to the binding and optional incorporation of the labeled nucleotides templated on the template sequences, and wherein a fluorescently labeled binder is attached to the binding moiety after the binding and optional incorporation and prior to the imaging. In any of the embodiments herein, the binding moiety/fluorescently labeled binder pair can comprise biotin, DNP, DIG, or desthiobiotin. In any of the embodiments herein, the binding moiety/fluorescently labeled binder pair comprises functional groups capable of reacting with each other, optionally wherein the functional groups are click functional groups.

In any of the embodiments herein, the binding moieties in the labeled nucleotides of the first and/or second pools can be non-fluorescent, and the labeled nucleotides of the first and/or second pools can become fluorescently detectable after the fluorescently labeled binders are attached to the corresponding binding moieties.

In any of the embodiments herein, the attachment of the fluorescently labeled binder to the binding moiety is reversible. For instance, desthiobiotin/biotin-binding can be dissociated by either biotin or desthiobiotin, e.g., free biotin or desthiobiotin can be used to compete with biotin labels (on nucleotides) bound to streptavidin-labeled fluorescent dyes, thereby dissociating the streptavidin-labeled fluorescent dyes from the nucleotides.

In any of the embodiments herein, each fluorescent label can be removable from the nucleotide attached thereto. In any of the embodiments herein, each fluorescent label can be cleavable from the nucleotide attached thereto. The cleavage can comprise chemical cleavage and/or enzymatic cleavage.

Also provided herein is a kit comprising: a) a first plurality of nucleotides comprises nucleotides of A, T/U, C, and G, wherein nucleotides of two of the four different bases comprise nucleotides labeled with a label detectable at a first wavelength, and wherein nucleotides of the remaining two of the four different bases comprise nucleotides labeled with a label detectable at a second wavelength different from the first wavelength; and b) a second plurality of nucleotides comprising nucleotides of A, T/U, C, and G, wherein nucleotides of one of the four different bases comprise nucleotides labeled with a label detectable at the first wavelength, wherein nucleotides of a different one of the four different bases comprise nucleotides labeled with a label detectable at the second wavelength different from the first wavelength, and wherein nucleotides of the remaining two of the four different bases are not detectably labeled; and c) optionally instructions for using the first and the second plurality of nucleotides according to the method in any one of the embodiments herein, wherein the first plurality of nucleotides is used as the first pool of nucleotides and the second plurality of nucleotides is used as the second pool of nucleotides, or vice versa. In some embodiments, (i) nucleotides of a particular base in the first plurality and (ii) nucleotides of the particular base in the second plurality are labeled with labels detectable at the first and second wavelengths, respectively, or wherein one of (i) and (ii) is not detectably labeled and other one of (i) and (ii) is labeled with a label detectable at the first or second wavelength.

Also provided herein is a kit comprising: a) a first plurality of nucleotides comprises nucleotides of A, T/U, C, and G, wherein nucleotides of two of the four different bases comprise nucleotides labeled with a label detectable at a first wavelength, wherein nucleotides of the remaining two of the four different bases comprise nucleotides labeled with a label detectable at a second wavelength different from the first wavelength, and wherein nucleotides of one of the remaining two different bases comprise nucleotides labeled with a label detectable at the first wavelength; and b) a second plurality of nucleotides comprising nucleotides of A, T/U, C, and G, wherein nucleotides of one of the four different bases comprise nucleotides labeled with a label detectable at the first wavelength or at the second wavelength, wherein nucleotides of the remaining three of the four different bases are not detectably labeled; and c) optionally instructions for using the first and the second plurality of nucleotides according to the method in in any one of the embodiments herein, wherein the first plurality of nucleotides is used as the first pool of nucleotides and the second plurality of nucleotides is used as the second pool of nucleotides, or vice versa. In some embodiments, the nucleotides of one of the remaining two different bases in a) comprise a nucleotide molecule labeled with a label detectable at the first wavelength and with a label detectable at the second wavelength. In any of the preceding embodiments, the nucleotides of one of the remaining two different bases in a) can comprise: a first nucleotide molecule labeled with a label detectable at the first wavelength; and a second nucleotide molecule labeled with a label detectable at the second wavelength.

In any of the preceding embodiments, nucleotides of A, T/U, C, and G can each independently comprise between about 5% and about 50% nucleotide molecules that are not detectably labeled. In any of the preceding embodiments, nucleotides of A, T/U, C, and G can each independently comprise about 10% nucleotide molecules that are not detectably labeled.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides an exemplary flowchart for nucleic acid sequencing, according to some embodiments described herein.

FIG. 2 provides exemplary nucleotide pools for the first and second nucleotide incorporations, according to some embodiments described herein. The nucleotides for the short reaction and the nucleotides for the long reaction can be switched in order.

FIG. 3 provides an exemplary schematic illustrating the first and second nucleotide incorporation at four clusters, according to some embodiments described herein.

FIG. 4 provides an exemplary truth table of detectable colors for each of the incorporated nucleotide labels after the first and second nucleotide incorporations, according to some embodiments described herein.

FIG. 5 provides exemplary nucleotide pools for the first and second nucleotide incorporations, according to some embodiments described herein. The nucleotides for the first incorporation and the nucleotides for the second incorporation can be switched in order.

FIG. 6 provides an exemplary schematic illustrating the first and second nucleotide incorporation at four clusters, according to some embodiments described herein.

FIG. 7 provides an exemplary truth table of detectable colors for each of the incorporated nucleotide labels after the first and second nucleotide incorporations, according to some embodiments described herein.

FIG. 8 provides an exemplary process for nucleic acid sequencing. In this example, sequencing-by-synthesis can be performed using four labelled nucleotides for a short incorporation (e.g., 2 seconds) such that within a clonal cluster, nucleotide incorporation occurs only at some strands within the cluster, whereas the other strands in the cluster do not have incorporation of the labeled nucleotides (that is, nucleotide incorporation at these strands is delayed or lagging behind within the cluster). A) shows representative status of DNA strands with primer during sequencing with ATGC as next base; B) shows the extended base after contact with dATP, dCTP, dGTP and dTTP with fluorescent tags, where the incorporation time is very short which only allow extension of only a subset of sequencing primers in the same cluster, for all clusters each having one of the four bases; C) shows a second incorporation step after contact with dTTP-G3, dATP-R3, dCTP and dGTP with long incorporation time (e.g., 30 seconds), such that the strands lagging behind within a cluster can have nucleotide incorporation (which can be incorporation of predominantly unlabeled nucleotides) in order to catch up with strands that have undergone nucleotide incorporation in the short (e.g., 2 seconds) incorporation; D) shows the 3′ blocker cleavage and next sequencing cycle; E) shows the truth table for base calling. The nucleotides for Incorporation 1 and the nucleotides for Incorporation 2 can be switched in order.

FIG. 9 provides an exemplary scheme showing streptavidin with dual dye labels which can be used to stain biotin or desthiobiotin tagged nucleotide during sequencing to improve the brightness of dual coded nucleotide such as T in FIG. 5 (right panel).

FIG. 10 provides an exemplary scheme showing a streptavidin mixture with different dye labels which can be used to stain biotin or desthiobiotin tagged nucleotide during sequencing to improve the brightness of dual coded nucleotides.

### DETAILED DESCRIPTION

Disclosed herein are methods and systems that overcome nucleotide incorporation inefficiencies during nucleic acid sequencing. Existing methods for nucleic acid sequencing rely extensively on labelled nucleotides. Such nucleotides often consist of at least one fluorescent molecule conjugated to the nucleotide base, and this added bulk promotes steric hindrance and inefficient reactions. Inefficient nucleotide incorporation during nucleic acid sequencing reactions can be the basis for several additional problems for nucleic acid sequencing methods, such as, but not limited to, slower sequencing reaction times, higher incidence of out-of-phase sequencing reactions, and more expensive reaction costs. To address the issues that arise from inefficient nucleotide incorporation, methods of the present disclosure employ an alternative scheme, so that unlabeled nucleotides are predominantly used during nucleic acid sequencing. The methods of the present disclosure address inefficient nucleotide incorporation during nucleic acid sequencing, and by extension, yield faster, cheaper, and more robust reactions.

The methods of the present disclosure detail a novel nucleotide coding scheme that enables the predominant use of unlabeled ‘dark’ nucleotides. In contrast to existing methods, which are often limited to one dark nucleotide base, the methods of the present disclosure can support the usage of two or three unlabeled nucleotide bases. The methods of the present disclosure use two rounds of nucleotide incorporation for each nucleotide position to be sequenced, wherein the first round is a brief pulse of a labeled nucleotide pool that does not saturate the template strand clusters, and the second round is a prolonged presentation of a nucleotide pool comprising two or three unlabeled nucleotide bases, which saturates the template strand clusters at the given nucleotide position. The two rounds of nucleotide incorporation together constitute a sequence or codeword of colors representative of each nucleotide base. The predominant use of unlabeled nucleotides in the methods of the present disclosure increases the efficiency of sequencing reactions over existing methods.

In some instances, for example, the disclosed methods for nucleic acid sequencing and nucleotide composition may comprise: a) contacting a substrate having clusters of polynucleotides immobilized thereon with a first pool of nucleotides; b) allowing binding and optional incorporation of the nucleotides of the first pool templated on only a subset of the multiple copies of the template sequence at each cluster; c) imaging the substrate to detect signals or absence thereof at the clusters; d) contacting the substrate with a second pool of nucleotides comprising at least two different bases and labeled nucleotides configured to be detected at one or more wavelengths; e) allowing binding and optional incorporation of the nucleotides of the second pool templated on the template sequence at each cluster; and f) imaging the substrate to detect signals or absence thereof at the clusters.

### I. Definitions

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

The terms “detectable label,” “optical label,” and “label” are used interchangeably herein to refer to a directly or indirectly detectable moiety that is coupled to or may be coupled to another moiety, for example, a nucleotide or nucleotide analog. The detectable label can be directly detectable by itself (e.g., radioisotope labels or fluorescent labels) or, in the case of an enzymatic label, can be indirectly detectable, e.g., by cataly zing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

In some embodiments, a detectable label is or includes a fluorophore. Exemplary fluorophores include, but are not limited to, fluorescent nanocrystals; quantum dots; d-Rhodamine acceptor dyes including dichloro[R110], dichloro[R6G], dichloro[TAMRA], dichloro[ROX] or the like; fluorescein donor dye including fluorescein, 6-FAM, or the like; Cyanine dyes such as Cy3B; Alexa dyes, SETA dyes, Atto dyes such as atto 647N which forms a FRET pair with Cy3B and the like. Fluorophores include, but are not limited to, MDCC (7-diethylamino-3-[([(2-maleimidyl)ethyl]amino)carbonyl]coumarin), TET, HEX, Cy3, TMR, ROX, Texas Red, Cy5, LC red 705 and LC red 640.

In some embodiments, a detectable label is or includes a luminescent or chemiluminescent moiety. Common luminescent/chemiluminescent moieties include, but are not limited to, peroxidases such as horseradish peroxidase (HRP), soy bean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy-and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “primer” is a single-stranded nucleic acid sequence having a 3′end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. Enzymatic extension can be performed by an enzyme including, but not limited to, a polymerase and/or a reverse transcriptase. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule.

Furthermore, a poly (dT) sequence may be used as a sequencing primer for sequencing RNA molecules comprising poly(A) tails.

A “non-terminating nucleotide” or “incorporating nucleotide” can include a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In some embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g., DNA polymerase activity of the wild-type sequence. Mutations can affect the activity profile of the enzymes, e.g., enhance or reduce the rate of polymerization, under different reaction conditions, e.g. temperature, template concentration, primer concentration, etc. Mutations or sequence-modifications can also affect the exonuclease activity and/or thermostability of the enzyme.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENTIM DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, Array Script™, MultiScribe™ ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g., M-MLV, and compositions including unmodified and modified enzymes are commercially available, e.g., Array Script™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g., an AMV or MMLV reverse transcriptase.

### II. Overview

Disclosed herein are methods and systems that overcome nucleotide incorporation inefficiencies during nucleic acid sequencing. Existing methods for nucleic acid sequencing rely extensively on labelled nucleotides. Such nucleotides often consist of at least one fluorescent molecule conjugated to the nucleotide base, and the added bulk promotes steric hindrance and inefficient reactions. Inefficient nucleotide incorporation during nucleic acid sequencing reactions can be the basis for several additional problems for nucleic acid sequencing methods, such as, but not limited to, slower sequencing reaction times, higher incidence of out-of-phase sequencing reactions, and more expensive reaction costs. To address the issues that arise from inefficient nucleotide incorporation, methods of the present disclosure employ an alternative coding scheme, so that unlabeled nucleotides are predominantly used during nucleic acid sequencing. The methods of the present disclosure address inefficient nucleotide incorporation during nucleic acid sequencing, and by extension, yield faster, cheaper, and more robust reactions.

Sequencing methods often require the disambiguation of the nucleotide bases, A, T, C, and G. In some existing methods, a straightforward coding scheme of a unique label per nucleotide base is used. For example, the nucleotide A may be conjugated to a label that is detectable at one wavelength, the nucleotide T may be conjugated to a label that is detectable at a second non-overlapping wavelength, the nucleotide C may be conjugated to a label that is detectable at a third non-overlapping wavelength, and the nucleotide G may be conjugated to a label that is detectable at a fourth non-overlapping wavelength. In such a coding scheme, all nucleotides are labelled. Consequently, such a scheme is one of the least efficient, in terms of nucleotide incorporation. Every nucleotide base is subject to extensive degrees of steric hindrance and poor incorporation reaction kinetics during DNA sequencing.

An alternative coding scheme may improve nucleotide incorporation efficiencies by representing one nucleotide as a non-labelled ‘dark’ nucleotide. For example, one coding scheme may comprise of the nucleotide A conjugated to a label that is detectable at one wavelength, the nucleotide T conjugated to a label that is detectable at a second non-overlapping wavelength, the nucleotide C conjugated to a label that is detectable at a third non-overlapping wavelength, and the nucleotide G not conjugated to a label. In such a coding scheme, the nucleotide G can be distinguished from the other nucleotides, because it does not emit any detectable light upon excitation. Three nucleotide bases are subject to relatively poor incorporation reaction kinetics during DNA sequencing. The unlabeled nucleotide base is not.

Another alternative coding scheme can improve on the scheme above, by using fewer types of labels, such as two labels, instead of three or four labels, to distinguish between the four nucleotides. This arguable simplicity can be achieved by generating a sequence or codeword representation. For example, the nucleotide A can be denoted with the presence of labels 1 and 2, the nucleotide T can be denoted by the presence of label 1 only, the nucleotide C can be denoted by the presence of label 2 only, and the nucleotide G can be denoted by the absence of any labels. Experimental implementations of codeword representations often use multiple labels for a nucleotide base. For example, in order to denote the nucleotide A with labels 1 and 2, either a mixture of As conjugated to label 1 and As conjugated to label 2 can be used, or/and As conjugated to both labels 1 and 2 can be used. Coding schemes that use only two types of labels also have an advantage over coding schemes that use more than two types of labels, because if using only two labels, only two images need to be acquired, when sequencing a given nucleotide-an image resulting from a detectable wavelength for label 1, and an image resulting from a detectable non-overlapping wavelength for label 2. In addition, the use of an unlabeled nucleotide to represent one of the nucleotide bases-such as an unlabeled G in the example above-helps with nucleotide incorporation efficiency.

The methods of the present disclosure provide an efficient coding scheme for sequencing nucleic acid molecules. In some embodiments, the nucleic acid sequences are determined by contacting the nucleic acid molecules with a first pool of nucleotides, allowing binding and optional incorporation of nucleotides in the first pool of nucleotides, imaging to detect signals (or absence thereof) associated with the bound and optionally incorporated nucleotides, contacting the nucleic acid molecules with a second pool of nucleotides, allowing binding and optional incorporation of nucleotides in the second pool of nucleotides, and imaging to detect signals (or absence thereof) associated with the bound and optionally incorporated nucleotides. In some embodiments, the nucleotides of the first pool of nucleotides are bound and optionally incorporated to a subset of the nucleic acid molecules, and the nucleotides of the second pool of nucleotides are bound and optionally incorporated to a majority of nucleic acid molecules.

In some embodiments, the methods of the present disclosure can use the acquisition of two or more images when sequencing a given nucleotide. In some embodiments, the methods of the present disclosure make extensive use of unlabeled nucleotides, such that two or three unlabeled nucleotides are used for the majority of the sequencing reactions. To do so, the methods of the present disclosure employ two rounds of nucleotide incorporation. The first round of incorporation pulses the nucleotides for a brief duration, such that only a subset of the template strands in the polynucleotide clusters are bound by nucleotides and incorporated into the synthesizing strands. The nucleotides from the first round of incorporation will often consist of only labeled nucleotides and will be inefficiently incorporated. Inefficient incorporation is acceptable, if not desirable, for the first incorporation round, because the goal of the round is to not saturate the template strands. The second round of incorporation, in contrast, will often comprise mostly of unlabeled nucleotides—at least two of the nucleotide bases will be unlabeled—and will be incorporated into the synthesizing strands relatively efficiently. Efficient incorporation is desirable for the second incorporation round, because the goal of the round is to saturate the template strands.

The dominant use of efficiently incorporated unlabeled nucleotides by methods in the present disclosure can provide several advantages. First, the total reaction time per cycle can, at least, be comparable or shorter than existing chemistries, even after consideration of a washing step between nucleotide incorporation rounds, and a cleavage step of 3′blockers.

The total reaction time per sequencing cycle can typically be 35 seconds, wherein a 2 second first nucleotide incorporation round is followed by three 1 second wash steps, which is followed by a 20 second long second nucleotide incorporation round, which is followed by a 10 second cleavage reaction time (2+3*1+20+10=35 seconds per cycle). A 35 second reaction time per cycle is faster than some current sequencing chemistries on the market, such as the NextSeq sequencing platform, which has a 60 second reaction time per cycle, and the MiniSeq Rapid Recipe sequencing platform, which has a 39 second reaction time per cycle. The efficient sequencing times provided by the methods of the present disclosure provide greater savings, as more sequencing cycles are performed for a given sequencing run. Given that a sequencing run can often comprise of several sequencing cycles, the methods of the present disclosure can provide immense savings in time and related instrument usage costs. Notably, the 35 second per cycle reaction time does not represent the most efficient estimate. The methods of the present disclosure can comprise a second nucleotide incorporation time of 10 seconds per cycle, as opposed to the 20 second long estimate provided in the example above, which would yield a 25 second per cycle reaction time.

In addition to shortened sequencing reaction times, the efficient incorporation of nucleotides can also deter a common issue among sequencing platforms known as ‘phasing’. Phasing refers to the event where at a given nucleotide index being sequenced, not all the template strands are bound by nucleotides. Sequencing then proceeds to the next cycle, but because the preceding nucleotide index of some strands was not bound by nucleotides, not all template strands in a cluster are being sequenced at the same index. As a result, at a given cluster of template strands, some template strands will lag behind other template strands, in their sequencing, and a mixed signal can be observed from a cluster, although a homogenous signal should be expected. The methods of the present disclosure can be less prone to phasing issues, because of their prevalent use of unlabeled nucleotides, which are efficiently incorporated. Template strands are less likely to not be bound by nucleotides, because most nucleotides in the methods of the present disclosure are likely unlabeled.

Last, the prevalent use of unlabeled nucleotides reduces the total cost of reagents used for sequencing reactions. Unlabeled nucleotides are cheaper than labeled nucleotides, and existing methods do not predominantly use unlabeled nucleotides, unlike the methods of the present disclosure. As a result, the methods of the present disclosure can reduce sequencing costs.

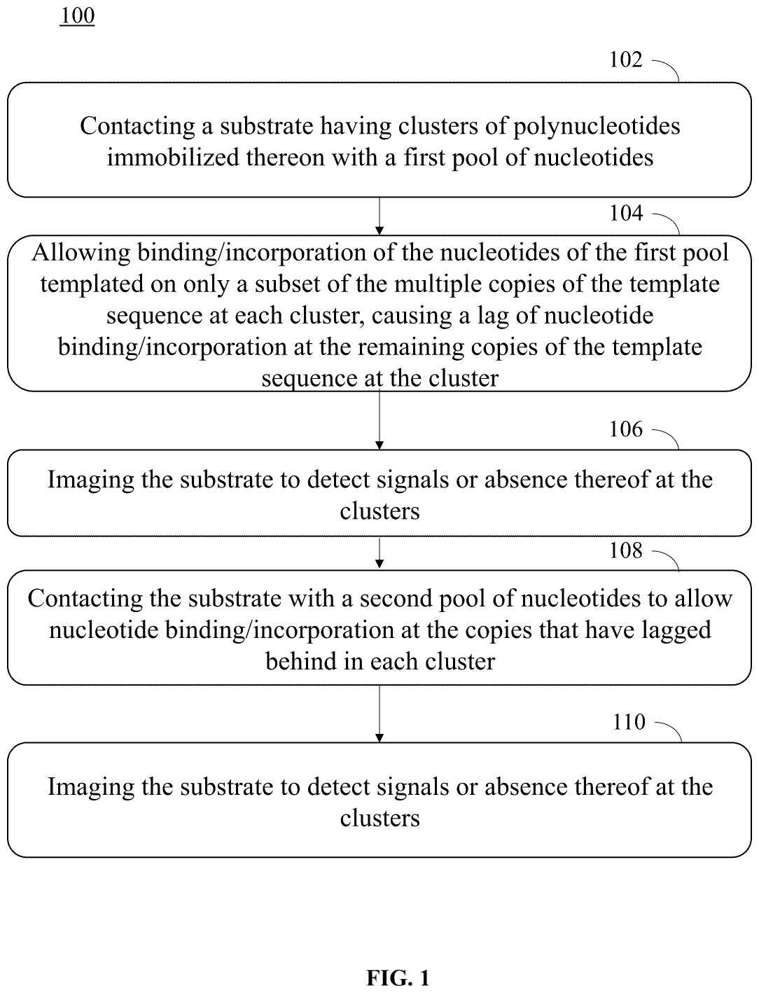

FIG. 1 illustrates an exemplary schematic showing a general process 100 for nucleic acid sequencing. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 102 in FIG. 1, a substrate having clusters of polynucleotides immobilized thereon can be contacted with a first pool of nucleotides, wherein each cluster comprises multiple copies of one of the different template sequences, and an oligonucleotide primer annealed to a primer binding sequence for extension of the oligonucleotide primer templated on a copy of the template sequence. The first pool of nucleotides can comprise at least two different bases, and labeled nucleotides configured to be detected at different wavelengths.

In some instances, the clusters can be disposed at spatially discrete sites on the substrate. In some instances, the average distance between adjacent clusters on the substrate can be between about 0.3 μm and about 10 μm. In some instances, the density of clusters on the substrate can be between about 100 k/mm² and about 5000 k/mm². In some instances, the signals from adjacent clusters on the substrate can be optically resolvable. In some instances, the clusters can comprise a random array of clusters on the substrate. In some instances, the clusters can comprise an ordered array of clusters on the substrate. In some instances, the clusters can be formed via bridge amplification. In some instances, the one or more clusters can each comprise multiple molecules, each comprising i) one or more adapter sequences and/or one or more primer binding sequences and ii) the same template sequence or complement thereof. In some instances, one or more of the clusters can be formed via rolling circle amplification (RCA). In some instances, the one or more clusters can each comprise one or more RCA products (RCPs), each comprising: i) one or more adapter sequences and/or one or more primer binding sequences and ii) the same template sequence. In some instances, each cluster can comprise i) one or more polynucleotides that are 5′ immobilized on the substrate and 3′ blocked; ii) one or more polynucleotides that are 3′ immobilized on the substrate; and/or iii) one or more nucleic acid concatemers immobilized on the substrate.

At step 104 in FIG. 1, the method allows binding and optional incorporation of the nucleotides of the first pool templated on only a subset of the multiple copies of the template sequence at each cluster, wherein the bound and optionally incorporated nucleotides are complementary to nucleotides at a first nucleotide position in the template sequences.

In some instances, the first pool of nucleotides can comprise nucleotides of any two or three of A, T/U, C, and G or nucleotides of A, T/U, C, and G. In some instances, the first pool of nucleotides comprises i) nucleotide molecules of the same base each labeled with or configured to be labeled with two or more different detectable labels; or ii) nucleotide molecules of the same base, wherein different subsets of the nucleotide molecules are each labeled with or configured to be labeled with two or more different detectable labels. In some instances, the first pool of nucleotides comprises labeled nucleotides configured to be detected at two different wavelengths, optionally wherein the first pool of nucleotides comprises nucleotides comprises nucleotides of one or two different bases that are not configured to be detected. In some instances, the first pool of nucleotides comprises one or more dNTP monomers and/or one or more dNTP multimers each comprising multiple dNTP molecules conjugated to a scaffold. In some instances, on average, no more than about 90%, about 80%, about 70%, about 60%, about 50%, about 40%, about 30%, about 20%, or about 10% of the multiple copies of the template sequence at each cluster are used as templates for the binding and optional incorporation of the nucleotides of the first pool. In some instances, on average, about 50% of the multiple copies of the template sequence at each cluster are used as templates for the binding and optional incorporation of the nucleotides of the first pool. In some instances, the clusters are allowed to contact with the first pool of nucleotides for no more than about 18, about 15, about 12, about 9, about 6 or about 3 seconds. In some instances, the clusters are allowed to contact with the first pool of nucleotides for about 2 seconds. In some instances, the first pool of nucleotides each comprises a reversible terminator, optionally wherein the reversible terminator is a 3′-O-blocked reversible terminator or a 3′-unblocked reversible terminator.

At step 106 in FIG. 1, the substrate can be imaged to detect signals or absence thereof at the clusters, wherein the signals are associated with the bound and optionally incorporated nucleotides of the first pool.

In some instances, the methods of the present disclosure can comprise removing unbound or unincorporated nucleotides from the substrate prior to imaging the substrate above. In some instances, the imaging can comprise imaging the substrate using two different fluorescent channels.

At step 108 in FIG. 1, the substrate can be contacted with a second pool of nucleotides comprising at least two different bases and labeled nucleotides configured to be detected at one or more wavelengths.

In some instances, the second pool of nucleotides comprises nucleotides of any two or three of A, T/U, C, and G, or comprises nucleotides of A, T/U, C, and G. In some instances, the second pool of nucleotides comprises labeled nucleotides configured to be detected at two different wavelengths. In some instances, the second pool of nucleotides comprises i) nucleotide molecules of the same base each labeled with or configured to be labeled with two or more different detectable labels; or ii) nucleotide molecules of the same base, wherein different subsets of the nucleotide molecules are each labeled with or configured to be labeled with two or more different detectable labels. In some instances, the second pool of nucleotides comprises nucleotides of one, two, or three different bases that are not configured to be detected. In some instances, the second pool of nucleotides comprises one or more dNTP monomers and/or one or more dNTP multimers each comprising multiple dNTP molecules conjugated to a scaffold. In some instances, the second pool of nucleotides each comprises a reversible terminator, optionally wherein the reversible terminator is 3′-O-blocked reversible terminator or a 3′-unblocked reversible terminator. In some instances, the labeled nucleotides in the first pool and/or the labeled nucleotides in the second pool are fluorescently labeled with one or more fluorophores prior to the binding and optional incorporation of the labeled nucleotides templated on the template sequences. In some instances, the labeled nucleotides in the first pool and/or the labeled nucleotides in the second pool can each be labeled with a binding moiety prior to the binding and optional incorporation of the labeled nucleotides templated on the template sequences, and wherein a fluorescently labeled binder is attached to the binding moiety after the binding and optional incorporation and prior to the imaging. In some instances, the binding moiety/fluorescently labeled binder pair can comprise biotin, DNP, DIG, or desthiobiotin. In some instances, the binding moiety/fluorescently labeled binder pair can comprise functional groups capable of reacting with each other, optionally wherein the functional groups are click functional groups. In some instances, each fluorescent label can be cleavable from the nucleotide attached thereto.

The methods can allow binding and optional incorporation of the nucleotides of the second pool templated on the template sequence at the copies that have lagged behind in each cluster, wherein the bound and optionally incorporated nucleotides are complementary to nucleotides at the first nucleotide position in the template sequences.

In some instances, the clusters can be allowed to contact with the second pool of nucleotides until substantially all of remaining copies of the template sequence at each cluster are used as templates for the binding and optional incorporation of the nucleotides of the second pool. In some instances, the clusters can be allowed to contact with the second pool of nucleotides for at least about 20, at least about 25, or about 30 seconds or longer. In some instances, the clusters can be allowed to contact with the second pool of nucleotides for about 30 seconds.

At step 110 in FIG. 1, the substrate can be imaged to detect signals or absence thereof at the clusters, wherein the signals are associated with the bound and optionally incorporated nucleotides of the second pool, wherein for each cluster, a signal codeword comprising signal codes corresponding to the signals or absence thereof detected is generated, wherein different signal codewords correspond to different bases, thereby determining the bases at the first nucleotide position in the plurality of polynucleotide templates.

In some instances, the methods of the present disclosure can comprise removing unbound or unincorporated nucleotides from the substrate prior to imaging the substrate. In some instances, the imaging can comprise imaging the substrate using one or two different fluorescent channels. In some instances, each different signal codeword corresponds to one of A, T/U, C, and G. In some instances, the methods of the present disclosure can comprise determining the bases at a second nucleotide position in the plurality of polynucleotide templates, wherein the second nucleotide position is 5′ to the first nucleotide position in the plurality of polynucleotide templates.

The second nucleotide position that is 5′ to the first nucleotide position can be interrogated/sequenced essentially as described for interrogating/sequencing the first nucleotide position. In some embodiments, the second nucleotide position can be sequenced by contacting the substrate with a first pool of nucleotides comprising: i) at least two different bases in the nucleotides of the first pool, and ii) labeled nucleotides configured to be detected at different wavelengths; allowing binding and optional incorporation of the nucleotides of the first pool templated on only a subset of the multiple copies of the template sequence at each cluster, wherein the bound and optionally incorporated nucleotides are complementary to nucleotides at the second nucleotide position (which is 5′ to the first nucleotide position) in the template sequences; imaging the substrate to detect signals or absence thereof at the clusters, wherein the signals are associated with the bound and optionally incorporated nucleotides of the first pool; contacting the substrate with a second pool of nucleotides comprising: i) at least two different bases in the nucleotides of the second pool, and ii) labeled nucleotides configured to be detected at one or more wavelengths; allowing binding and optional incorporation of the nucleotides of the second pool templated on the template sequence at each cluster, wherein the bound and optionally incorporated nucleotides are complementary to nucleotides at the second nucleotide position in the template sequences; and imaging the substrate to detect signals or absence thereof at the clusters, wherein the signals are associated with the bound and optionally incorporated nucleotides of the second pool, wherein for each cluster, a signal codeword comprising signal codes corresponding to the signals or absence thereof detected in the imaging steps is generated, wherein different signal codewords correspond to different bases, thereby determining the bases at the second nucleotide position in the plurality of polynucleotide templates. In some embodiments, the second nucleotide position can be sequenced after removing 3′ blocking groups of the nucleotide residues incorporated at the first nucleotide position in the template sequences.

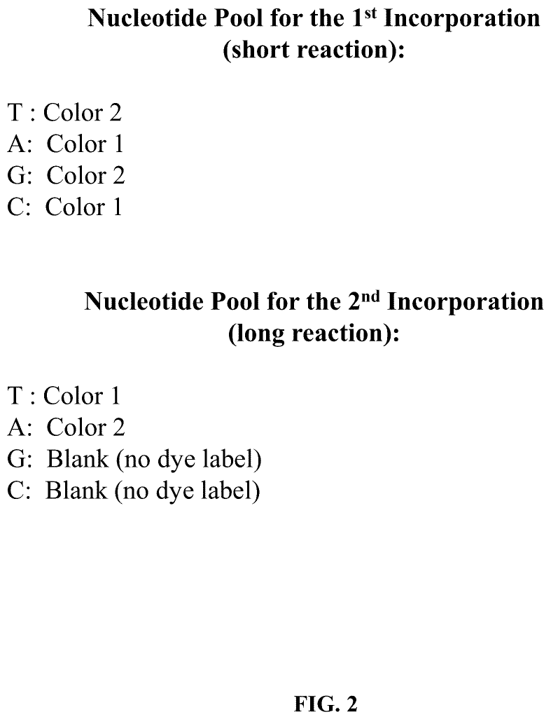

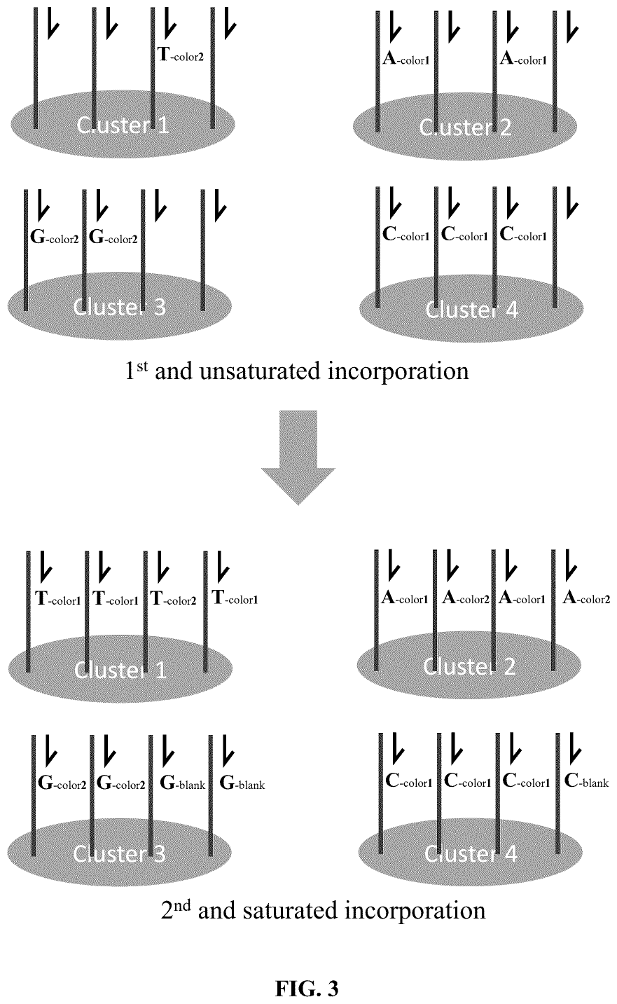

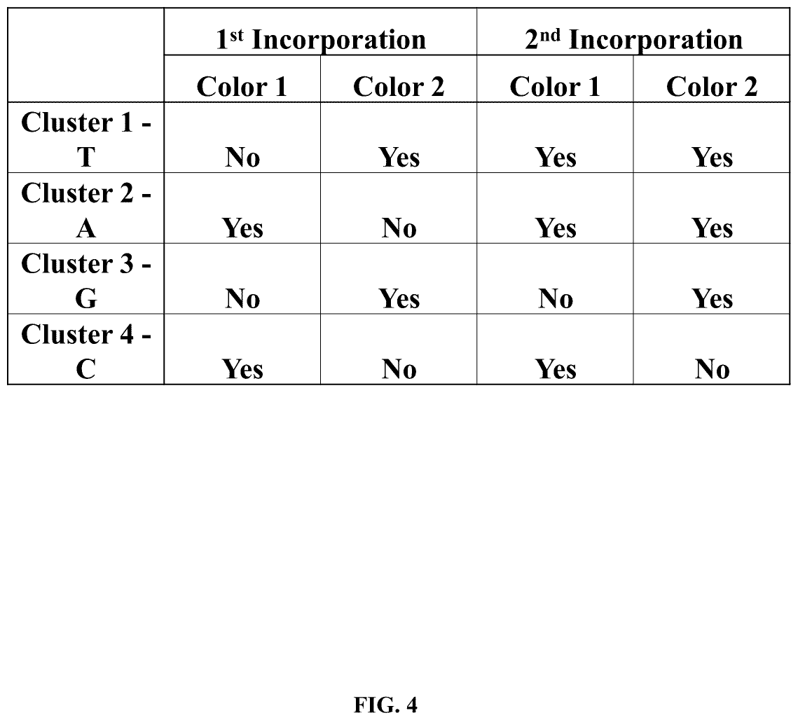

FIGS. 2-4 illustrate a non-limiting exemplary coding scheme and schematic process by which two unlabeled nucleotide bases are used for the majority of the sequencing reactions, according to some methods of the present disclosure. As depicted in FIG. 2, if two unlabeled nucleotide bases are used for the majority of the sequencing reactions, then each nucleotide base of the first nucleotide label set is labelled with one of two colors. The second nucleotide label set comprises of two unlabeled nucleotide bases, and two labeled nucleotide bases. As depicted in FIG. 3, a subset of the template strands of each cluster is bound by the first nucleotide label set, such as the one seen in FIG. 2, and incorporated into the synthesizing strands; some strands are not bound by nucleotides at the position being sequenced. At least one image is then acquired at the wavelengths detectable for both labels. Then, during the second nucleotide incorporation, the second nucleotide label set, such as the one seen in FIG. 2, which comprises unlabeled Gs and Cs, binds to the remaining template strands at each cluster, such that all the template strands are bound by nucleotides, at the position being sequenced. At least one image is then acquired at the wavelengths detectable for both labels. The resulting output of the acquired images is a dictionary of codewords, wherein each codeword comprises a sequence of colors and represents a nucleotide base. FIG. 4 shows an exemplary dictionary of codewords for nucleotide bases, wherein each codeword is derived from nucleotide label sets and sequencing reactions, such as, but not limited to, those shown in FIGS. 2 and 3, respectively.

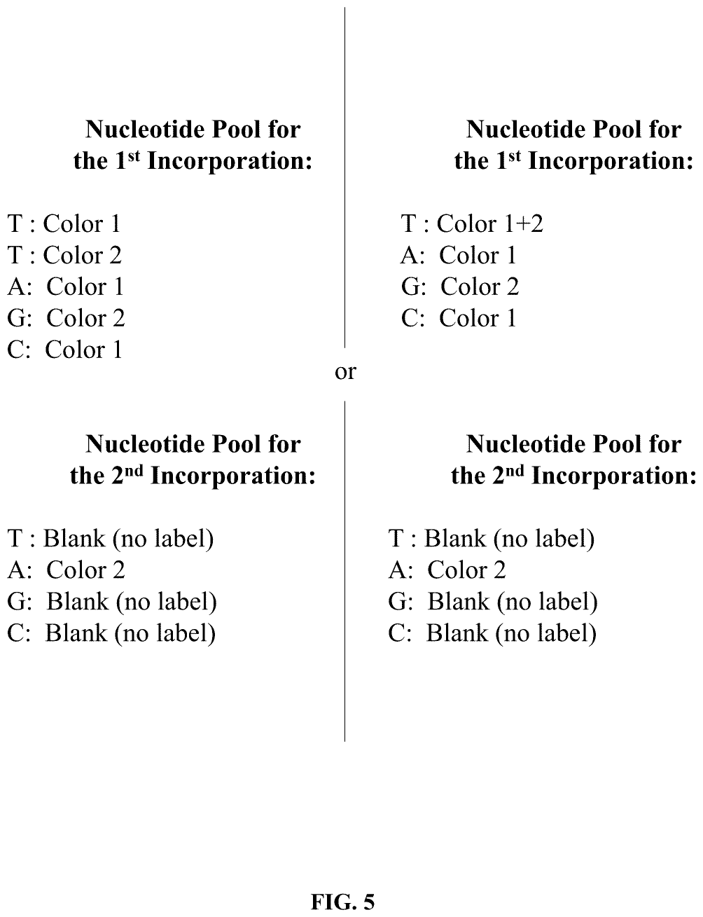

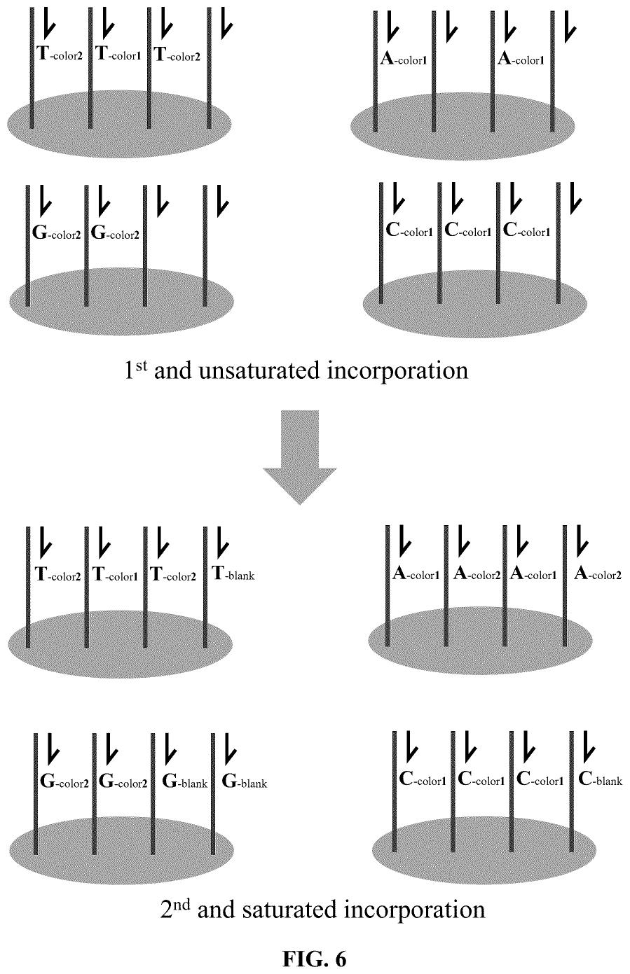

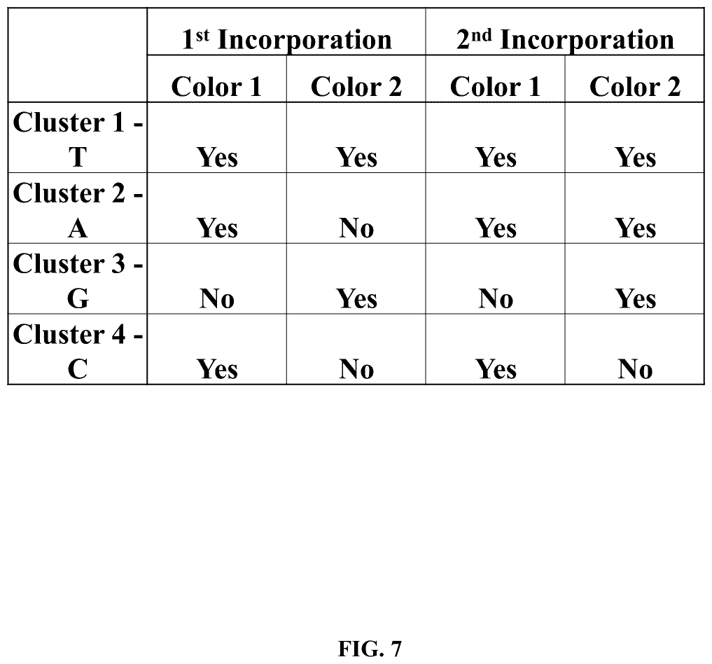

FIGS. 5-7 illustrate a non-limiting exemplary coding scheme and schematic process by which three unlabeled nucleotide bases are used for the majority of the sequencing reactions, according to some methods of the present disclosure. As depicted in FIG. 5, if three unlabeled nucleotide bases are used for the majority of the sequencing reactions, then three of the four nucleotide bases of the first nucleotide label set are labelled with one of two colors. The fourth nucleotide base is labelled with both colors, either by having both labels bound to the nucleotide base simultaneously, or/and by having a mixed population containing some proportion of the fourth nucleotide labeled with the first color, and the other proportion of the fourth nucleotide labeled with the second color. As depicted in FIG. 6, a subset of the template strands of each cluster is bound by the first nucleotide label set, such as the one seen in FIG. 5, and incorporated into the synthesizing strands; some strands are not bound by nucleotides at the position being sequenced. At least one image is then acquired at the wavelengths detectable for both labels. Then, during the second nucleotide incorporation, the second nucleotide label set, such as the one seen in FIG. 5, which comprises unlabeled Ts, Gs, and Cs, binds to the remaining template strands at each cluster, such that all the template strands are bound by nucleotides at the position being sequenced. At least one image is then acquired at the wavelengths detectable for both labels. The resulting output of the acquired images is a dictionary of codewords, wherein each codeword comprises a sequence of colors and represents a nucleotide base. FIG. 7 shows an exemplary dictionary of codewords for nucleotide bases, wherein each codeword is derived from nucleotide label set and sequencing reactions, such as, but not limited to, those shown in FIGS. 5 and 6, respectively.

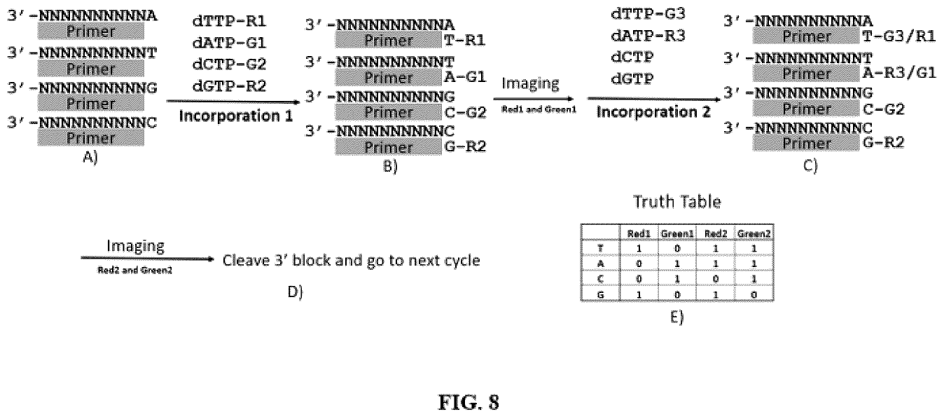

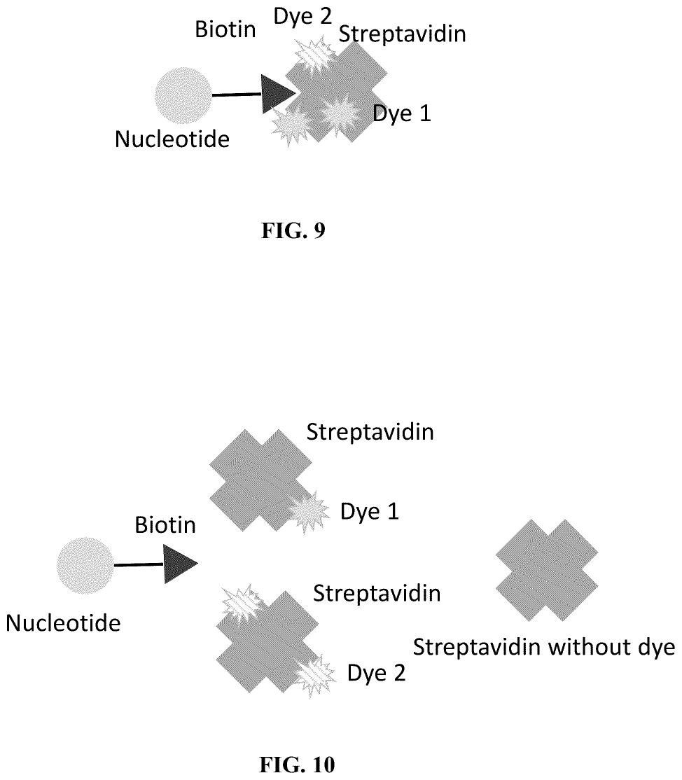

In another base coding method, as shown in FIG. 8, all four nucleotides are labelled with detectable tags. In this case, dTTP is labelled with red dye (R1). dATP is labelled with green dye (G1). dCTP is with green dye (G2) and dGTP is labelled with red dye (R2). The first incorporation mixture has very brief contact (e.g. 2 s) with DNA clusters on surface which allow only part of extendable sites is extended with polymerase and dNTP. An image step is employed after proper wash steps. Two images are acquired with green and red excitation source and dual band filters. T and G clusters are detectable in red image. A and C clusters are detectable in green image. The second incorporation mixture is introduced with dTTP-G3 (green dye), dATP-R3 (red dye), dCTP (dark) and dGTP (dark). The incorporation time of 2nd mixture is much longer than 1st incorporation (e.g. 30 s) which allow full extension of all extendable sites. The growing lag (phasing) can be significantly improved since dark nucleotides can be used in the mixture. In FIG. 8, dCTP and dGTP can be 100% without labels. However, dTTP and dATP without tags can also be mixed with dTTP-G3 and dATP-R3 to improve the phasing during sequencing. This four-base coding algorithm can significantly improve the accuracy of base calling result since these four images provided 16 possible states in true table and only 4 of them are used for four bases. Any error status or miss called cluster in image processing can be detected and potential corrected.

### III. Samples and Nucleic Acid Molecules

The nucleic acid molecules used in the methods described herein may be obtained from any suitable biological source, for example a tissue sample, a blood sample, a plasma sample, a saliva sample, a fecal sample, or a urine sample. The polynucleotides may be DNA or RNA molecules. In some embodiments, RNA molecules are reverse transcribed into DNA molecules prior to hybridizing the polynucleotide to a sequencing primer. In some embodiments, RNA molecules are not reverse transcribed and are hybridized to a sequencing primer for direct RNA sequencing. In some embodiments, the nucleic acid molecule is a cell-free DNA (cfDNA), such as a circulating tumor DNA (ctDNA) or a fetal cell-free DNA.

Examples of nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid molecules also include RNA molecules such as various types of coding and non-coding RNA, including viral RNAs. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the nucleic acid molecules disclosed herein are non-capped mRNA, a non-poly adenylated mRNA, and a non-spliced mRNA. The RNA analyte can be a transcript of another nucleic acid molecule (e.g., DNA or RNA such as viral RNA).

In some embodiments, a nucleic acid molecule may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In some embodiments, a nucleic acid molecule can be extracted from a cell, a virus, or a tissue sample comprising the cell or virus. Processing conditions can be adjusted to extract or release nucleic acid molecules (e.g., RNA) from a cell, a virus, or a tissue sample.

### IV. Sequencing Methods

### A. Nucleotides and Nucleotide Analogs

In some embodiments, a method disclosed herein comprises using one or more nucleotides or analogs thereof, including a native nucleotide or a nucleotide analog or modified nucleotide (e.g., labeled with one or more detectable labels). In some embodiments, a nucleotide analog comprises a nitrogenous base, five-carbon sugar, and phosphate group, wherein any component of the nucleotide may be modified and/or replaced. In some embodiments, a method disclosed herein may comprise but does not require using one or more non-incorporable nucleotides. Non-incorporable nucleotides may be modified to become incorporable at any point during the sequencing method.

Nucleotide analogs include, but are not limited to, alpha-phosphate modified nucleotides, alpha-beta nucleotide analogs, beta-phosphate modified nucleotides, beta-gamma nucleotide analogs, gamma-phosphate modified nucleotides, caged nucleotides, or ddNTPs. Examples of nucleotide analogs are described in U.S. Pat. No. 8,071,755, which is incorporated by reference herein in its entirety.

In some embodiments, a method disclosed herein may comprise but does not require using terminators that reversibly prevent nucleotide incorporation at the 3′-end of the primer. One type of reversible terminator is a 3′-O-blocked reversible terminator. Here the terminator moiety is linked to the oxygen atom of the 3′-OH end of the 5-carbon sugar of a nucleotide. For example, U.S. Pat. Nos. 7,544,794 and 8,034,923 (the disclosures of these patents are incorporated by reference) describe reversible terminator dNTPs having the 3′-OH group replaced by a 3′-ONH2 group. Another type of reversible terminator is a 3′-unblocked reversible terminator, wherein the terminator moiety is linked to the nitrogenous base of a nucleotide. For example, U.S. Pat. No. 8,808,989 (the disclosure of which is incorporated by reference) discloses particular examples of base-modified reversible terminator nucleotides that may be used in connection with the methods described herein. Other reversible terminators that similarly can be used in connection with the methods described herein include those described in U.S. Pat. Nos. 7,956,171, 8,071,755, and 9,399,798, herein incorporated by reference.

In some embodiments, a method disclosed herein may comprise but does not require using nucleotide analogs having terminator moieties that irreversibly prevent nucleotide incorporation at the 3′-end of the primer. Irreversible nucleotide analogs include 2′, 3′-dideoxynucleotides, ddNTPs (ddGTP, ddATP, ddTTP, ddCTP). Dideoxynucleotides lack the 3′-OH group of dNTPs that is essential for polymerase-mediated synthesis.

In some embodiments, a method disclosed herein may comprise but does not require using non-incorporable nucleotides comprising a blocking moiety that inhibits or prevents the nucleotide from forming a covalent linkage to a second nucleotide (3′-OH of a primer) during the incorporation step of a nucleic acid polymerization reaction. The blocking moiety can be removed from the nucleotide, allowing for nucleotide incorporation.

In some embodiments, a method disclosed herein may comprise but does not require using 1, 2, 3, 4 or more nucleotide analogs present in the SBS reaction. In some embodiments, a nucleotide analog is replaced, diluted, or sequestered during an incorporation step. In some embodiments, a nucleotide analog is replaced with a native nucleotide. In some embodiments, a nucleotide analog is modified during an incorporation step. The modified nucleotide analog can be similar to or the same as a native nucleotide.

In some embodiments, a method disclosed herein may comprise but does not require using a nucleotide analog having a different binding affinity for a polymerase than a native nucleotide. In some embodiments, a nucleotide analog has a different interaction with a next base than a native nucleotide. Nucleotide analogs and/or non-incorporable nucleotides may base-pair with a complementary base of a template nucleic acid.

In some embodiments, one or more nucleotides can be labeled with distinguishing and/or detectable tags or labels. The tags may be distinguishable by means of their differences in fluorescence, Raman spectrum, charge, mass, refractive index, luminescence, length, or any other measurable property. The tag may be attached to one or more different positions on the nucleotide, so long as the fidelity of binding to the polymerase-nucleic acid complex is sufficiently maintained to enable identification of the complementary base on the template nucleic acid correctly. In some embodiments, the tag is attached to the nucleobase of the nucleotide. Alternatively, a tag is attached to the gamma phosphate position of the nucleotide.

Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to another moiety, for example, a nucleotide or nucleotide analog, and can include a fluorescent, a colorimetric, or a chemiluminescent label.

In some embodiments, a detectable label can be attached to another moiety, for example, a nucleotide or nucleotide analog. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor®350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocy anine, JC-1, 6-JOE, JOJO™-1/JO-PRO™-1, LDS 751 (+DNA), LDS 751 (+RNA), LOLOT-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTOR 11, SYTOR 13, SYTOR 17, SYTOR 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′IRDye® 700, 5′IRDye® 800, 5′IRDye® 800CW (NHS Ester), WellRED D4 Dye, WellRED D3 Dye, WellRED D2 Dye, Lightcycler® 640 (NHS Ester), and Dy 750 (NHS Ester).

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

### B. Polymerases

Polymerases that may be used to carry out the disclosed techniques include naturally-occurring polymerases and any modified variations thereof, including, but not limited to, mutants, recombinants, fusions, genetic modifications, chemical modifications, synthetics, and analogs. Naturally occurring polymerases and modified variations thereof are not limited to polymerases that retain the ability to catalyze a polymerization reaction. In some embodiments, the naturally occurring and/or modified variations thereof retain the ability to catalyze a polymerization reaction. In some embodiments, the naturally-occurring and/or modified variations have special properties that enhance their ability to sequence DNA, including enhanced binding affinity to nucleic acids, reduced binding affinity to nucleic acids, enhanced catalysis rates, reduced catalysis rates, etc. Mutant polymerases include polymerases wherein one or more amino acids are replaced with other amino acids (naturally or non-naturally occurring), and insertions or deletions of one or more amino acids.

In some embodiments, a method disclosed herein may comprise but does not require using modified polymerases containing an external tag (e.g., an exogenous detectable label), which can be used to monitor the presence and interactions of the polymerase. In some embodiments, intrinsic signals from the polymerase can be used to monitor their presence and interactions. Thus, the provided methods can include monitoring the interaction of the polymerase, nucleotide and template nucleic acid through detection of an intrinsic signal from the polymerase. In some embodiments, the intrinsic signal is a light scattering signal. For example, intrinsic signals include native fluorescence of certain amino acids such as tryptophan.

In some embodiments, a method disclosed herein may comprise using an unlabeled polymerase, and monitoring is performed in the absence of an exogenous detectable label associated with the polymerase. Some modified polymerases or naturally occurring polymerases, under specific reaction conditions, may incorporate only single nucleotides and may remain bound to the primer-template after the incorporation of the single nucleotide.

In some embodiments, a method disclosed herein may comprise using an polymerase unlabeled with an exogenous detectable label (e.g., a fluorescent label). The label can be chemically linked to the structure of the polymerase by a covalent bond after the polymerase has been at least partially purified using protein isolation techniques. For example, the exogenous detectable label can be chemically linked to the polymerase using a free sulfhydryl or a free amine moiety of the polymerase. This can involve chemical linkage to the polymerase through the side chain of a cysteine residue, or through the free amino group of the N-terminus. In certain preferred embodiments, a fluorescent label attached to the polymerase is useful for locating the polymerase, as may be important for determining whether or not the polymerase has localized to a spot on an array corresponding to immobilized primed template nucleic acid. The fluorescent signal need not, and in some embodiments does not change absorption or emission characteristics as the result of binding any nucleotide. In some embodiments, the signal emitted by the labeled polymerase is maintained uniformly in the presence and absence of any nucleotide being investigated as a possible next correct nucleotide.

The term polymerase and its variants, as used herein, also refers to fusion proteins comprising at least two portions linked to each other, for example, where one portion comprises a peptide that can cataly ze the polymerization of nucleotides into a nucleic acid strand is linked to another portion that comprises a second moiety, such as, a reporter enzyme or a processivity-modifying domain. For example, T7 DNA polymerase comprises a nucleic acid polymerizing domain and a thioredoxin binding domain, wherein thioredoxin binding enhances the processivity of the polymerase. Absent the thioredoxin binding, T7 DNA polymerase is a distributive polymerase with processivity of only one to a few bases.

Although DNA polymerases differ in detail, they have a similar overall shape of a hand with specific regions referred to as the fingers, the palm, and the thumb; and a similar overall structural transition, comprising the movement of the thumb and/or finger domains, during the synthesis of nucleic acids.

DNA polymerases include, but are not limited to, bacterial DNA polymerases, eukaryotic DNA polymerases, archaeal DNA polymerases, viral DNA polymerases and phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, ϵ, η, ζ, λ, σ, μ, and κ, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cp7 DNA polymerase, T7 DNA polymerase, and T4 polymerase. Other DNA polymerases include thermostable and/or thermophilic DNA polymerases such as DNA polymerases isolated from Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. Engineered and modified polymerases also are useful in connection with the disclosed techniques. For example, modified versions of the extremely thermophilic marine archaea Thermococcus species 9° N (e.g., Therminator DNA polymerase from New England BioLabs Inc. ; Ipswich, Mass.) can be used. Still other useful DNA polymerases, including the 3PDX polymerase are disclosed in U.S. Pat. No. 8,703,461, the disclosure of which is incorporated by reference in its entirety.

RNA polymerases include, but are not limited to, viral RNA polymerases such as T7 RNA polymerase, T3 polymerase, SP6 polymerase, and K11 polymerase; Eukaryotic RNA polymerases such as RNA polymerase I, RNA polymerase II, RNA polymerase III, RNA polymerase IV, and RNA polymerase V; and Archaea RNA polymerase.

Reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase that maintains the telomeres of eukaryotic chromosomes.

### C. Sequencing Reactions

In some embodiments of a sequencing-by-synthesis (SBS) method disclosed herein, a first labeled nucleotide that has been incorporated is not deactivated (e.g., by removal and/or photobleaching of the label) prior to the introduction and/or incorporation of the next, second labeled nucleotide. The first and second labeled nucleotides can comprise the same base or different bases. The first and second labeled nucleotides can be introduced into a sequencing reaction mix simultaneously or at different time points in any order. Further, the first and second labeled nucleotides can be introduced by itself (e.g., in a suitable solvent such as water) or in a mixture with another sequencing reagent, such as one or more other labeled nucleotides and/or one or more unlabeled nucleotides. The first and second labeled nucleotides can also comprise the same base or different bases. In some embodiments, nucleotides that have not been incorporated at a residue corresponding to a base in the template nucleic acid (e.g., because the first labeled nucleotide has been incorporated at that residue) are not removed from the sequencing reaction mix prior to the introduction and/or incorporation of the second labeled nucleotide. In some embodiments, the first and second labeled nucleotides (and optionally labeled nucleotides for interrogating subsequent bases in the template) are provided in the same sequencing reaction mix, and the first, second, and optionally any subsequent labeled nucleotide(s) are incorporated sequentially in a continuous manner.

Thus, unlike existing SBS methods, some embodiments of the method disclosed herein use continuous introduction and/or incorporation of nucleotides (e.g., fluorescently labeled A, T, C, and/or G nucleotides) without the need of label deactivation and/or wash steps in between sequential incorporation events for a given template nucleic acid molecule to be sequenced. Rather, in some embodiments, label deactivation (e.g., by cleaving and/or photobleaching the label) of a first incorporated nucleotide may occur stochastically throughout the continuous nucleotide incorporation process, for instance, prior to, during, or after the incorporation of a second, third, fourth, or a subsequent labeled nucleotide.

Nucleic acid sequencing reaction mixtures, or simply “reaction mixtures,” typically include reagents that are commonly present in polymerase based nucleic acid synthesis reactions. The reaction mixture can include other molecules including, but not limited to, enzymes. In some embodiments, the reaction mixture comprises any reagents or biomolecules generally present in a nucleic acid polymerization reaction. Reaction components may include, but are not limited to, salts, buffers, small molecules, detergents, crowding agents, metals, and ions. In some embodiments, properties of the reaction mixture may be manipulated, for example, electrically, magnetically, and/or with vibration.

The provided methods herein may further comprise but do not require one or more wash steps; a temperature change; a mechanical vibration; a pH change; or an optical stimulation that is not dye illumination or photobleaching. In some embodiments, the wash step comprises contacting the substrate and the nucleic acid molecule, the primer, and/or the polymerase with one of more buffers, detergents, protein denaturants, proteases, oxidizing agents, reducing agents, or other agents capable of crosslinking or releasing crosslinks, e.g., crosslinks within a polymerase or crosslinks between a polymerase and nucleic acid. Methods and compositions for nucleic acid sequencing are known, for example, as described in U.S. Pat. Nos. 10,246,744 and 10,844,428, incorporated herein by reference in their entireties for all purposes.

Reaction mixture reagents can include, but are not limited to, enzymes (e.g., polymerase), dNTPs, template nucleic acids, primer nucleic acids, salts, buffers, small molecules, co-factors, metals, and ions. The ions may be catalytic ions, divalent catalytic ions, non-catalytic ions, non-covalent metal ions, or a combination thereof. The reaction mixture can include salts, such as NaCl, KCl, potassium acetate, ammonium acetate, potassium glutamate, or NH4Cl or the like, that ionize in aqueous solution to yield monovalent cations. The reaction mixture can include a source of ions, such as Mg²⁺, Mn²⁺, Co²⁺, Cd²⁺, and/or Ba²⁺ ions. The reaction mixture can include tin, Ca²⁺, Zn²⁺, Cu²⁺, Co²⁺, Fe²⁺, and/or Ni²⁺, or other divalent non-catalytic metal cations. In some embodiments, the reaction mixture can include metal cations that may inhibit formation of phosphodiester bonds between the primed template nucleic acid molecule and the cognate nucleotide. In some embodiments, the metal cations can be used (e.g., at a suitable concentration) to slow down but not completely inhibit or prevent nucleotide incorporation, thereby reducing multiple nucleotide incorporation events in a single detection window.

In some embodiments, the sequencing reaction conditions comprise contacting the nucleic acid molecule and the primer with a buffer that regulates osmotic pressure. In some embodiments, the reaction mixture comprises a buffer that regulates osmotic pressure. In some embodiments, the buffer is a high salt buffer that includes a monovalent ion, such as a monovalent metal ion (e.g., potassium ion or sodium ion) at a concentration of from about 50 to about 1,500 mM. Salt concentrations in the range of from about 100 to about 1,500 mM, or from about 200 to 1,000 mM may also be used. In some embodiments, the buffer further comprises a source of glutamate ions (e.g., potassium glutamate). In some embodiments, the buffer comprises a stabilizing agent. In some embodiments, the stabilizing agent is a non-catalytic metal ion (e.g., a divalent non-catalytic metal ion). Non-catalytic metal ions useful in this context include, but are not limited to, calcium, strontium, scandium, titanium, vanadium, chromium, iron, cobalt, nickel, copper, zinc, gallium, germanium, arsenic, selenium, rhodium, europium, and/or terbium. In some embodiments, the non-catalytic metal ion is strontium, tin, or nickel. In some embodiments, the sequencing reaction mixture comprises strontium chloride or nickel chloride. In some embodiments, the stabilizing agent can be used (e.g., at a suitable concentration) to slow down but not completely inhibit or prevent nucleotide incorporation, thereby reducing multiple nucleotide incorporation events in a single detection window.

The buffer can include Tris, Tricine, HEPES, MOPS, ACES, MES, phosphate-based buffers, and acetate-based buffers. The reaction mixture can include chelating agents such as EDTA, EGTA, and the like. In some embodiments, the reaction mixture includes cross-linking reagents.

In some embodiments, the interaction between the polymerase and template nucleic acid may be manipulated by modulating sequencing reaction parameters such as ionic strength, pH, temperature, or any combination thereof, or by the addition of a destabilizing agent to the reaction. In some embodiments, the destabilizing agent can be used (e.g., at a suitable concentration) to slow down but not completely inhibit or prevent nucleotide incorporation, thereby reducing multiple nucleotide incorporation events in a single detection window.

In some embodiments, high salt (e.g., 50 to 1,500 mM) and/or pH changes are utilized to destabilize a complex between the polymerase and template nucleic acid. In some embodiments, the reaction conditions favor the stabilization of a complex among the polymerase, the template nucleic acid, and a labeled nucleotide. By way of example, the pH of the reaction mixture can be adjusted from 4.0 to 10.0 to favor the stabilization of a complex among the polymerase, the template nucleic acid, and a labeled nucleotide. In some embodiments, the pH of the reaction mixture is from 4.0 to 6.0. In some embodiments, the pH of the reaction mixture is 6.0 to 10.0. In some embodiments, a suitable salt concentration and/or a suitable pH can be selected to slow down but not completely inhibit or prevent nucleotide incorporation, thereby reducing multiple nucleotide incorporation events in a single detection window.

In some embodiments, the reaction mixture comprises a competitive inhibitor, where the competitive inhibitor may reduce the occurrence of multiple incorporations events in a detection window. In one embodiment, the competitive inhibitor is a non-incorporable nucleotide. In an embodiment, the competitive inhibitor is an aminoglycoside. The competitive inhibitor is capable of replacing either the nucleotide or the catalytic metal ion in the active site, such that the competitive inhibitor occupies the active site preventing or slowing down a nucleotide incorporation. In some embodiments, both an incorporable nucleotide and a competitive inhibitor are introduced, such that the ratio of the incorporable nucleotide and the inhibitor can be adjusted to modulate the rate of incorporation of a single nucleotide at the 3′-end of the primer. In some embodiments, the competitive inhibitor can be used (e.g., at a low concentration) to slow down but not completely inhibit or prevent nucleotide incorporation, thereby reducing multiple nucleotide incorporation events in a single detection window.

In some embodiments, the reaction mixture comprises at least one nucleotide molecule that is a non-incorporable nucleotide. In some embodiments, the reaction mixture comprises one or more nucleotide molecules incapable of incorporation into the primer of the primed template nucleic acid molecule. Such nucleotides incapable of incorporation include, for example, monophosphate nucleotides. For example, the nucleotide may contain modifications to the triphosphate group that make the nucleotide non-incorporable. Examples of non-incorporable nucleotides may be found in U.S. Pat. No. 7,482,120, which is incorporated by reference herein in its entirety. In some embodiments, the primer may not contain a free hydroxyl group at its 3′-end, thereby rendering the primer incapable of incorporating any nucleotide, and, thus, making any nucleotide non-incorporable. In some embodiments, the primer may be processed such that it contains a free hydroxyl group at its 3′-end to allow nucleotide incorporation. In some embodiments, the non-incorporable nucleotide can be used (e.g., at a low concentration) to slow down but not completely inhibit or prevent nucleotide incorporation, thereby reducing multiple nucleotide incorporation events in a single detection window.

In some embodiments, the reaction mixture comprises at least one nucleotide molecule that is incorporable but is incorporated at a slower rate compared to a corresponding naturally-occurring nucleoside triphosphate (e.g., NTP or dNTP). Such nucleotides incorporable at a slower rate may include, for example, diphosphate nucleotides. For example, the nucleotide may contain modifications to the triphosphate group that make the nucleotide incorporable at a slower rate. In some embodiments, the nucleotide incorporable at a slower rate can be used to slow down but not completely inhibit or prevent nucleotide incorporation, thereby reducing multiple nucleotide incorporation events in a single detection window.

In some embodiments, the reaction mixture comprises a polymerase inhibitor. In some embodiments, the polymerase inhibitor is a pyrophosphate analog. In some embodiments, the polymerase inhibitor is an allosteric inhibitor. In some embodiments, the polymerase inhibitor is a DNA or an RNA aptamer. In some embodiments, the polymerase inhibitor competes with a catalytic-ion binding site in the polymerase. In some embodiments, the polymerase inhibitor is a reverse transcriptase inhibitor. The polymerase inhibitor may be an HIV-1 reverse transcriptase inhibitor or an HIV-2 reverse transcriptase inhibitor. The HIV-1 reverse transcriptase inhibitor may be a (4/6-halogen/MeO/EtO-substituted benzo[d]thiazol-2-yl)thiazolidin-4-one. In some embodiments, the polymerase inhibitor can be used (e.g., at a low concentration) to slow down but not completely inhibit or prevent nucleotide incorporation, thereby reducing multiple nucleotide incorporation events in a single detection window.

In some embodiments, the contacting step is facilitated by the use of a chamber such as a flow cell. The methods and apparatus described herein may employ next generation sequencing technology (NGS), which allows massively parallel sequencing. In some embodiments, single DNA molecules are sequenced in a massively parallel fashion within a reaction chamber. A flow cell may be used but is not necessary. Flowing liquid reagents through the flow cell, which contains an interior solid support surface (e.g., a planar surface), conveniently permits reagent exchange. Immobilized to the interior surface of the flow cell is one or more primed template nucleic acids to be sequenced or interrogated using the procedures described herein. Typical flow cells will include microfluidic valving that permits delivery of liquid reagents (e.g., components of the “reaction mixtures” discussed herein) to an entry port. Liquid reagents can be removed from the flow cell by exiting through an exit port.

In some embodiments, a reaction chamber disclosed herein can comprise a reagent wall, an imaging area, and optionally an outlet configured to remove molecules of one or more of the polymerase, the first detectably labeled nucleotide, the second detectably labeled nucleotide, and/or one or more other reagents from the imaging area. In some embodiments, the device may comprise one or more vents but no outlet or exit port for the reaction mixture. In some embodiments, a method disclosed herein does not comprise a step of removing liquid reagents through an outlet or exit port, e.g., from a reaction chamber such as a flow cell.

The methods disclosed herein may but do not need to be used in combination with any NGS sequencing methods. The sequencing technologies of NGS include but are not limited to pyrosequencing, sequencing-by-synthesis with reversible dye terminators, sequencing by oligonucleotide probe ligation, and ion semiconductor sequencing. Nucleic acids such as DNA or RNA from individual samples can be sequenced individually (singleplex sequencing) or nucleic acids such as DNA or RNA from multiple samples can be pooled and sequenced as indexed genomic molecules (multiplex sequencing) on a single sequencing run, to generate up to several hundred million reads of sequences. Examples of sequencing technologies that can be used to obtain the sequence information according to the present method are further described here.

Some sequencing technologies are available commercially, such as the sequencing-by-synthesis platforms from 454 Life Sciences (Bradford, Conn.), Illumina/Solexa (Hayward, Calif.) and Helicos Biosciences (Cambridge, Mass.).

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM).

In some embodiments, the disclosed methods may be used in combination with massively parallel sequencing of nucleic acid molecules using Illumina's sequencing-by-synthesis and reversible terminator-based sequencing chemistry. In some implementation, a method disclosed herein can use a flow cell having a glass slide with lanes.

After sequencing of nucleic acid molecules, sequence reads of predetermined length, e.g., at least about 15 bp, are localized by mapping (alignment) to a known reference sequence or genome (e.g., viral sequences or genomes). A number of computer algorithms are available for aligning sequences, including without limitation BLAST, BLITZ, FASTA, BOWTIE, or ELAND (Illumina, Inc., San Diego, Calif., USA).

In some embodiments, the provided sequencing methods disclosed herein may regulate polymerase interaction with the nucleotides and template nucleic acid (as well as rate of nucleotide incorporation) in a manner that reveals the identity of the next base while controlling the chemical addition of a nucleotide. In some embodiments, the SBS reaction condition comprises a plurality of primed template nucleic acids, polymerases, nucleotides, or any combination thereof. In some embodiments, the plurality of nucleotides comprises 1, 2, 3, 4, or more types of different nucleotides, for example dATP, dTTP (or dUTP), dGTP, and dCTP.

In some embodiments, the method can further comprise contacting the nucleic acid molecule with the substrate to immobilize the nucleic acid molecule. In some embodiments, the nucleic acid molecule can be immobilized at a density of one molecule per at least about 250 nm², at least about 200 nm², at least about 150 nm², at least about 100 nm², at least about 90 nm², at least about 80 nm², at least about 70 nm², at least about 60 nm², at least about 50 nm², at least about 40 nm², at least about 30 nm², at least about 20 nm², at least about 10 nm², at least about 5 nm², or in between any two of the aforementioned values. Methods and compositions for arraying biomolecules on a substrate, e.g., as described in US 2005/0042649 (incorporated herein by reference in its entirety for all purposes), may be used in methods disclosed herein.

In some embodiments, a subset of nucleic acid molecules (e.g., nucleic acid strands to be sequenced) on the substrate may be active at one or more time points. In some embodiments, at any one time, a first subset of nucleic acid molecules on the substrate is active (e.g., allowing nucleotide incorporation into a sequencing primer using a single-stranded sequence as template) while a second subset of nucleic acid molecules on the substrate is inactive (e.g., not allowing nucleotide incorporation into a sequencing primer using a single-stranded sequence as template). In some embodiments, at one or more time points, a first subset of nucleic acid molecules on the substrate is activated (e.g., by a first set of polymerase and/or primer molecules) for nucleotide incorporation, while a second subset of nucleic acid molecules on the substrate is not activated (e.g., by the first set of polymerase and/or primer molecules), thus only signals associated with the first subset of nucleic acid molecules are detected. At one or more other time points, the second subset of nucleic acid molecules on the substrate is activated (e.g., by a second set of polymerase and/or primer molecules) for nucleotide incorporation, while the first subset of nucleic acid molecules on the substrate is not activated (e.g., by the second set of polymerase and/or primer molecules), thus only signals associated with the second subset of nucleic acid molecules are detected. In some embodiments, the first and second sets of polymerase and/or primer molecules can be introduced at different time points, e.g., in sequential cycles with optional washing steps between cycles (e.g., to remove a set of polymerase and/or primer molecules for SBS of a first subset of strands before introducing the next set of polymerase and/or primer molecules for SBS of a second subset of strands).

In some embodiments, the substrate can comprise a bead, a planar substrate, a solid surface, a flow cell, a semiconductor chip, a well, a pillar, a chamber, a channel, a through hole, a nanopore, or any combination thereof. In some embodiments, the substrate can comprise a microwell, a micropillar, a microchamber, a microchannel, or any combination thereof.

### V. Compositions, Kits, and Applications

Also provided herein are compositions and kits comprising one or more of the primers, nucleic acid molecules, substrates, nucleotides including detectably labeled nucleotides, polymerases, and reagents for performing the methods provided herein, for example reagents required for one or more steps comprising hybridization, ligation, amplification, detection, sequencing, and/or sample preparation as described herein, for example, in Section IV.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for sample processing, such as nucleic acid extraction, isolation, and/or purification, e.g., RNA extraction, isolation, and/or purification. In some embodiments, the kits contain reagents, such as enzymes and buffers for ligation and/or amplification, such as ligases and/or polymerases. In some embodiments, the kits contain reagents, such as enzymes and buffers for primer extension and/or nucleic acid sequencing, such as polymerases and/or transcriptases. In some aspects, the kit can also comprise any of the reagents described herein, e.g., buffer components for tuning the rate of nucleotide incorporation and/or for tuning the rate of signal deactivation (e.g., by photobleaching). In some embodiments, the kits contain reagents for signal detection during sequencing, such as detectable labels and detectably labeled molecules. In some embodiments, the kits optionally contain other components, for example nucleic acid primers, enzymes and reagents, buffers, nucleotides, modified nucleotides, and reagents for additional assays.

In some aspects, the provided embodiments can be applied in analyzing nucleic acid sequences, such as DNA and/or RNA sequencing. In some aspects, the embodiments can be applied in an imaging or detection method for multiplexed nucleic acid analysis. In some aspects, the provided embodiments can be used to identify or detect regions of interest in target nucleic acids, such as viral DNA or RNA. In some embodiments, the region of interest comprises one or more nucleotide residues, such as a single-nucleotide polymorphism (SNP), a single-nucleotide variant (SNV), substitutions such as a single-nucleotide substitution, mutations such as a point mutation, insertions such as a single-nucleotide insertion, deletions such as a single-nucleotide deletion, translocations, inversions, duplications, and/or other sequences of interest.

In some aspects, the embodiments can be applied in investigative and/or diagnostic applications, for example, for characterization or assessment of a sample from a subject. Applications of the provided method can comprise biomedical research and clinical diagnostics. For example, in biomedical research, applications comprise, but are not limited to, genetic and genomic analysis for biological investigation or drug screening. In clinical diagnostics, applications comprise, but are not limited to, detecting gene markers such as disease, immune responses, bacterial or viral DNA/RNA for patient samples, loss of genetic heterozygosity, the presence of gene alleles indicative of a predisposition towards disease or good health, likelihood of responsiveness to therapy, or in personalized medicine or ancestry.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for determining a template sequence of a polynucleotide template at a location on a substrate, wherein multiple copies of the template sequence are provided at the location, the method comprising:
a) contacting the substrate with a first pool of nucleotides comprising labeled nucleotides configured to be detected at different wavelengths;
b) allowing binding and optional incorporation of the nucleotides of the first pool on a first subset of the multiple copies of the template sequence at the location, wherein the bound and optionally incorporated nucleotides are complementary to nucleotide residues at a first nucleotide position in the template sequence;
c) imaging the substrate to detect a signal (or record an absence thereof) at the location at each of the different wavelengths, wherein the signals or absences thereof are associated with a bound and optionally incorporated nucleotide of the first pool at the location;
d) contacting the substrate with a second pool of nucleotides comprising labeled nucleotides configured to be detected at one or more of the different wavelengths;
e) allowing binding and optional incorporation of the nucleotides of the second pool on a second subset of the multiple copies of the template sequence at the location, wherein the first and second subsets are different subsets, and wherein the bound and optionally incorporated nucleotides are complementary to nucleotide residues at the first nucleotide position in the template sequence; and
f) imaging the substrate to detect a signal (or record an absence thereof) at the location at each of the different wavelengths, wherein the signals or absences thereof are associated with a bound and optionally incorporated nucleotide of the second pool at the location,
wherein for the location, a signal codeword comprising signal codes corresponding to the signals or absences thereof in c) and f) is generated, wherein the signal codeword corresponds to the identity of the base in the bound and optionally incorporated nucleotides, thereby identifying the nucleotide residue at the first nucleotide position in the template sequence.

2. The method of claim 1, wherein the polynucleotide template is in a cluster of multiple polynucleotides immobilized at the location on the substrate, wherein each polynucleotide in the cluster comprises one or more copies of the template sequence, wherein the substrate comprises a plurality of clusters immobilized thereon, and wherein each cluster comprises multiple polynucleotides immobilized at a spatially discrete location on the substrate.

3. The method of claim 2, wherein one or more clusters of the plurality of clusters are formed via bridge amplification.

4. The method of claim 1, wherein the polynucleotide template is a rolling circle amplification product immobilized on the substrate, wherein the rolling circle amplification product comprises multiple copies of the template sequence.

5. The method of claim 1, wherein the first pool of nucleotides comprises:
i) nucleotide molecules of the same base, wherein each nucleotide molecule is labeled with or configured to be labeled with two or more different detectable labels detectable at different wavelengths; or
ii) nucleotide molecules of the same base, wherein different subsets of the nucleotide molecules are each labeled with or configured to be labeled with two or more different detectable labels detectable at different wavelengths.

6. The method of claim 1, wherein the first pool of nucleotides comprises labeled nucleotides configured to be detected at two different wavelengths, and wherein the first pool of nucleotides comprises nucleotides of one or two different bases that are not configured to be detected.

7. The method of claim 1, wherein on average about 50% of the multiple copies of the template sequence at each location on the substrate are used as templates for the binding and optional incorporation of the nucleotides of the first pool.

8. The method of claim 1, wherein each location on the substrate is allowed to contact with the first pool of nucleotides for about 2 seconds.

9. The method of claim 1, wherein the first pool of nucleotides each comprises a reversible terminator.

10. The method of claim 1, wherein the second pool of nucleotides comprises:
i) nucleotide molecules of the same base, wherein each nucleotide molecule is labeled with or configured to be labeled with two or more different detectable labels detectable at different wavelengths; or
ii) nucleotide molecules of the same base, wherein different subsets of the nucleotide molecules are each labeled with or configured to be labeled with two or more different detectable labels detectable at different wavelengths.

11. The method of claim 1, wherein the second pool of nucleotides comprises nucleotides of one, two, or three different bases that are not configured to be detected.

12. The method of claim 1, wherein each location on the substrate is allowed to contact with the second pool of nucleotides until substantially all of remaining copies of the template sequence at the location are used as templates for the binding and optional incorporation of the nucleotides of the second pool.

13. The method of claim 1, wherein each location on the substrate is allowed to contact with the second pool of nucleotides for about 30 seconds.

14. The method of claim 1, wherein the second pool of nucleotides each comprises a reversible terminator.

15. The method of claim 1, wherein a different signal codeword is generated for each of a plurality of the different locations on the substrate, and each different signal codeword corresponds to one of A, T/U, C, and G.

16. The method of claim 1, wherein:
one or more nucleotides in the first pool and/or one or more nucleotides in the second pool are each fluorescently labeled with one or more fluorophores prior to the binding and optional incorporation templated on the template sequence; or
one or more nucleotides in the first pool and/or one or more nucleotides in the second pool are each labeled with a binding moiety prior to the binding and optional incorporation templated on the template sequence, and wherein a fluorescently labeled binder is attached to the binding moiety after the binding and optional incorporation and prior to the imaging.

17. The method of claim 1, wherein the first pool of nucleotides comprises nucleotides of A, T/U, C, and G, wherein nucleotides of two of the four different bases are labeled with a label detectable at a first wavelength, and wherein nucleotides of the remaining two of the four different bases are labeled with a label detectable at a second wavelength different from the first wavelength.

18. The method of claim 1, wherein the second pool of nucleotides comprises nucleotides of A, T/U, C, and G, wherein nucleotides of one of the four different bases are labeled with a label detectable at a first wavelength, wherein nucleotides of a different one of the four different bases are labeled with a label detectable at a second wavelength different from the first wavelength, and wherein nucleotides of the remaining two of the four different bases are not detectably labeled.

19. The method of claim 1, wherein the bound and optionally incorporated nucleotide of the first pool at the location in c) remains detectable in f).

20. A method for determining sequences of a plurality of polynucleotide templates having different template sequences, comprising:
a) contacting a substrate having clusters of polynucleotides immobilized thereon with a first pool of nucleotides and with oligonucleotide primers, in any order,
whereby each cluster comprises:
i) multiple copies of one of the different template sequences, and
ii) an oligonucleotide primer of the oligonucleotide primers annealed to a primer binding sequence for extension of the oligonucleotide primer templated on a copy of the template sequence of i),
wherein the first pool of nucleotides comprises:
i) at least two different bases in the nucleotides of the first pool, and
ii) labeled nucleotides configured to be detected at different wavelengths;
b) allowing binding and optional incorporation of the nucleotides of the first pool templated on only a subset of the multiple copies of the template sequence at each cluster, wherein the bound and optionally incorporated nucleotides are complementary to nucleotides at a first nucleotide position in the template sequences;
c) imaging the substrate to detect signals or absence thereof at the clusters, wherein the signals are associated with the bound and optionally incorporated nucleotides of the first pool;
d) contacting the substrate with a second pool of nucleotides comprising:
i) at least two different bases in the nucleotides of the second pool, and
ii) labeled nucleotides configured to be detected at one or more wavelengths;
e) allowing binding and optional incorporation of the nucleotides of the second pool templated on the template sequence at each cluster, wherein the bound and optionally incorporated nucleotides are complementary to nucleotides at the first nucleotide position in the template sequences; and
f) imaging the substrate to detect signals or absence thereof at the clusters, wherein the signals are associated with the bound and optionally incorporated nucleotides of the second pool,
wherein for each cluster, a signal codeword comprising signal codes corresponding to the signals or absence thereof detected in c) and f) is generated, wherein different signal codewords correspond to different bases, thereby determining the bases at the first nucleotide position in the plurality of polynucleotide templates.
